(19) United States
(12) Patent Application Publication
(10) Pub. No.: US 2024/0390245 A1
(43) Pub. Date: Nov. 28, 2024

(54) OXIDATIVE HAIR TREATMENT AGENT WITH A CONTROLLED HEAT DEVELOPMENT BASED ON ENVIRONMENTALLY FRIENDLY CONTENTS

(71) Applicant: Henkel AG & Co. KGaA, Duesseldorf (DE)

(72) Inventors: Bernd Anderheggen, Mönchengladbach (DE); Anja Reichert, Düsseldorf (DE); Konstantin Goutsis, Jüchen (DE)

(21) Appl. No.: 18/797,678

(22) Filed: Aug. 8, 2024

Related U.S. Application Data
(63) Continuation of application No. PCT/EP2023/050082, filed on Jan. 3, 2023.

(30) Foreign Application Priority Data
Feb. 10, 2022 (DE) 102022201371.9

(51) Int. Cl. A61K 8/44; A61Q 5/08

(52) U.S. Cl. CPC A61K 8/44; A61Q 5/08; A61K 2800/5922; A61K 2800/882

(57) ABSTRACT

The invention relates to oxidative hair treatment agents, in particular bleaching powder and bleaching pastes, comprising a content of biodegradable complexing agents which do not have a strong heating effect even when used on hair with a high metal or heavy metal content, thus gently lightening the hair or dyeing the hair in a lightening manner.

### FIELD OF INVENTION

The present invention relates to oxidative hair treatment agents—in particular, bleaching powder and bleaching pastes—comprising a content of biodegradable complexing agents that do not have a strong heating effect even when used on hair having a higher metal or heavy metal content, thus gently lightening the hair or dyeing the hair in a lightening manner.

The present invention further relates to the use of the agents for gentle bleaching or oxidative lightening or dyeing in a lightening manner of human hair, and to a multi-component packaging unit (kit-of-parts) for lightening keratin fibers or dyeing keratin fibers in a lightening manner, which comprises an oxidative hair treatment agent and, separately therefrom, an oxidizing agent preparation.

A method for oxidative lightening or dyeing in a lightening manner of keratin fibers using the aforementioned oxidative hair treatment agent and the kit comprising this oxidative hair treatment agent is also described.

The oxidative hair treatment agent according to the invention is a substantially anhydrous oxidizing agent preparation containing at least one persalt. The substantially anhydrous oxidative hair treatment agent is preferably powdery or pasty.

### BACKGROUND

Many consumers desire to lighten their own hair, as blond hair color is considered to be attractive and desirable from a fashion point of view. Various bleaching agents with different bleaching power are commercially available for this purpose. Oxidizing agents present in these products are capable of lightening hair fibers by oxidatively degrading the hair's own colorant, melanin. For a moderate bleaching effect, the use of hydrogen peroxide-optionally with use of ammonia or other alkalizing agents—as sole oxidizing agent is sufficient. In order to attain a stronger bleaching effect, a mixture of hydrogen peroxide and at least one compound, selected from persalts, and in particular peroxydisulfate salts and/or peroxymonosulfate salts, is usually used. To intensify the bleaching effect, the agents contain higher use concentrations of hydrogen peroxide and persalts, and in particular persulfates. Dark, dark brown, or black hair can thus be lightened by 4 to 6 shades in a single step. The hydrogen peroxide and hair treatment agent containing persalt are stored separately from one another until they are used so as not to prematurely deactivate the persalts. The hydrogen peroxide component, which comprises an aqueous solution of hydrogen peroxide, has an acidic pH value—in particular, a pH value of 1.5 to 5.5, and in particular of 3 to 5, measured in each case at 20° C., for stabilization of the hydrogen peroxide.

For the melanin-degrading effect of the hydrogen peroxide and the bleaching effect on the keratin fibers, it is advantageous, however, if the mixture to be used formed of hydrogen peroxide solution and hair treatment agent containing persalt has an alkaline pH value, which lies in the range of 8.0 to 11.5, preferably in the range of 8.5 to 11.0, particularly preferably in the range of 9.0 to 10.5, and exceptionally preferably in the range of 9.5 to 10.0, measured in each case at 20° C.

In order to set an alkaline pH value of the lightening mixture to be used, oxidative hair treatment agents preferred according to the invention can contain, in addition to the at least one persalt, at least one powdery alkalizing agent in such a total amount that the mixture to be used has the desired alkaline pH value. Alternatively, a third composition can be added to the mixture to be used of oxidative hair treatment agent according to the invention and hydrogen peroxide solution, which third composition contains one or more alkalizing agents in such an amount that the mixture to be used has an alkaline pH value, which lies in the range of 8.0 to 11.5, preferably in the range of 8.5 to 11.0, particularly preferably 9.0 to 10.5, and exceptionally preferably 9.5 to 10.3, measured in each case at 20° C.

If oxidation dye precursors and/or direct dyes are added to the oxidative hair treatment agent, the hair can be dyed at the same time. Corresponding hair dyes are offered in particular for consumers having very dark, melanin-rich hair.

A requirement for the oxidative hair treatment agent according to the invention was to damage the hair as little as possible despite the harsh chemical conditions that prevail during oxidative lightening, without impairing the quality of the lightening. The active ingredients used for this purpose should be as environmentally friendly as possible.

The use of complexing agents in a higher concentration for reducing hair damage in oxidative hair treatment was already known from WO2002089754A1, WO2002074272A1, and WO2002074273A1. EP1714634A1 discloses a hair treatment kit for dyeing human hair, comprising a first compartment with a complexing agent and a second compartment with a dye. The complexing agent should prevent undesired reactions on and with hair that lead to undesired heating of the agent during the preparation of the mixture to be used and its time of action on the hair.

Many bleaching products on the market contain HEDP (1-hydroxyethane-1,1-diphosphonic acid, etidronic acid) salts thereof, or EDTA (ethylenediaminetetraacetic acid) or salts thereof. HEDP and EDTA stabilize hydrogen peroxide in an effective manner and complex metal ions present efficiently such that an undesired temperature increase taking place during application is virtually completely avoided. However, a major disadvantage of HEDP and EDTA is their poor biodegradability. In recent times, consumers increasingly consider the ecological profile of the cosmetic products used by them. For example, consumers prefer, in particular, cosmetics that are as sustainable as possible, containing biodegradable ingredients.

The use of substances of natural origin as complexing agents is also already known in the prior art. For example, FR2852834A1 discloses agents for bleaching, dyeing, or shaping hair, which contain the combination of at least one oxidizing agent and at least one polyhydroxycarboxylic acid. Even if the polyhydroxycarboxylic acids of FR2852834A1 are biodegradable, the performance properties of the agents described in this document are still in need of improvement, and especially with regard to heat development, which is particularly unfavorable in the presence of gluconic acid and salts thereof, and bleaching power.

EP1602356A1 discloses decolorizing agents or bleaching agents that contain the biodegradable complexing agent ethylenediamine-N,N′-disuccinic acid in combination with a persulfate.

However, the performance properties of the agents disclosed in the aforementioned documents are still in need of improvement, and in particular the bleaching power cannot yet be considered to be optimal. The temperature development occurring on the hair during the application also requires further optimization.

Another problem that can occur during the production of a bleaching agent is the swelling of the mixture to be used of the bleaching agent and the hydrogen peroxide-containing developer. Excessive foaming due to the presence of certain surfactants can also be problematic, and especially if the bleaching agent is applied to individual hair strands rather than the entire hair. In the case of strand bleaching, the strands to which the ready-to-use bleaching agent are applied are often wound into a film, or the strands are wound into films to which the bleaching agent is applied. The swelling of the agent and the development of foam reduce the contact surface of the bleaching agent with the hair and thus impair the bleaching result.

### SUMMARY OF THE INVENTION

It was the object of the present invention to find oxidative hair treatment agents, and in particular bleaching agents or agents for oxidative color change, containing biodegradable complexing agents, which are as superior as possible to the bleaching or lightening agents known from the prior art with regard to their bleaching performance. Furthermore, the bleaching agents should have a sufficiently high stability and, even when used on hair with a higher metal or heavy metal content, should not heat up too much. Furthermore, the swelling and/or the formation of foam of the ready-to-use lightening or bleaching agent should be contained as well as possible during the time of action on the hair.

These objects were achieved surprisingly by the subject matter of the claims. The complexing agents b)i), b)ii), and b)iii) are described as biodegradable and therefore represent an ecologically advantageous replacement for HEDP and EDTA. Surprisingly, it has been found in addition that the bleaching power could also be significantly improved using these special complexing agents (b) in the agent according to the invention.

According to the invention, keratin-containing or keratin fibers are understood in particular to mean human hair, but also fur, wool, and feathers.

### DETAILED DESCRIPTION OF THE INVENTION

A first subject matter of the present invention is an oxidative hair treatment agent for lightening hair or for dyeing hair in a lightening manner, containing:
a) at least one oxidizing agent selected from inorganic salts of a peroxysulfuric acid, and mixtures of these salts,
b) further, at least one complexing agent selected from
b) i) complexing agents of the general formula (I),

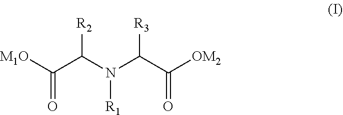

where
R1 represents a hydrogen atom, a C₁-C₆ alkyl group, a hydroxy-C₂-C₆ alkyl group, a carboxy-C₁-C₆ alkyl group, or a physiologically tolerated salt thereof,
R2, R3, independently of one another, represent a hydrogen atom, a C₁-C₆ alkyl group, a hydroxy-C₂-C₆ alkyl group, a carboxy-C₁-C₆ alkyl group, or a physiologically tolerated salt thereof, wherein R2 and R3 do not simultaneously represent a hydrogen atom and do not simultaneously represent a carboxy-C₁-C₆ alkyl group,
M1, M2, independently of one another, represent a hydrogen atom or an alkali metal ion—preferably sodium, potassium, or an ammonium ion (NH₄⁺)—and
b) ii) complexing agents of the general formula (II)

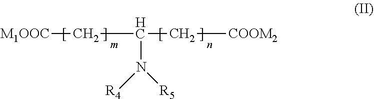

where
R4, R5, independently of one another, represent a carboxy-C₁-C₆ alkyl group or a physiologically tolerated salt thereof, a hydrogen atom, a C₁-C₆ alkyl group, or a hydroxy-C₂-C₆ alkyl group, and
m represents an integer from 0 to 6, and
n represents an integer from 0 to 6, and
M1, M2, independently of one another, represent a hydrogen atom or an alkali metal ion—preferably sodium, potassium, or an ammonium ion (NH₄⁺),
b) iii) ethylenediamine-N,N′-disuccinic acid or at least one alkali metal salt or ammonium salt of this acid,
c) further, at least one amino acid selected from alpha-amino acids, beta-amino acids, and the salts of an alpha-amino acid or beta-amino acid, and mixtures of these substances,
d) further, Polyquaternium-4, and
e) 0 to 10 wt %, preferably 0.1 to 8 wt %, and particularly preferably 0.5 to 5 wt % water, in each case relative to the weight of the oxidative hair treatment agent,
wherein no polymer is contained that comprises acrylic acid, acrylic acid amides, acrylic acid esters, methacrylic acid, methacrylic acid amides, and/or methacrylic esters as monomers.

The oxidative hair treatment agent, which is substantially anhydrous, with 0 to 10 wt % water, is preferably in powder, tablet, granular, or paste form.

According to the invention, the terms, “powder” or “powdery,” shall be understood to mean a pourable form of administration that is solid at 20° C. and 1,013 mbar and composed of individual particles, in which the individual particles have particle sizes in the range of 0.1 μm to no more than 1.6 mm. The particle size distribution can preferably be determined by way of laser diffraction measurement according to ISO 13320-1 (2009). Optionally, the grain size of the particles can be adapted to the requirements with regard to the bleaching powder by physical treatment such as sifting, pressing, granulating, or pelletizing, or by adding certain auxiliary substances, e.g., so as to enable better miscibility of the individual powder components, or the miscibility of the bleaching powder with a hydrogen peroxide preparation.

Preferred bleaching powders according to the invention have a bulk density in the range of 400 to 1,000 g/L (grams/liter), preferably 450 to 900 g/L, and particularly preferably 550 to 820 g/L. The bulk density is preferably determined according to EN ISO 60 (version January 2000) or DIN ISO 697 (version January 1984).

Unless specified otherwise, all specified temperatures relate to a pressure of 1,013 mbar.

The oxidative hair treatment agent according to the invention can also be in tablet form. For this purpose, a bleaching powder according to the invention is preferably pressed into a tablet. The oxidative hair treatment agent according to the invention can also be in granule form. For this purpose, a bleaching powder according to the invention is preferably processed to form granules.

The terms, “paste” or “paste-like,” are to be understood, according to the invention, to mean an administration form which, at 20° C. and 1,013 mbar, has a viscosity in the range of 200,000 to 1,600,000 mPas, preferably 250,000 to 1,400,000 mPas, particularly preferably 300,000 to 1,000,000 mPas, and exceptionally preferably 400,000 to 750,000 mPas.

The paste viscosity is preferably determined by means of Brookfield; apparatus RVDV II+; spindle no. 96, 4 revolutions per minute, at 20° C.

As a first essential component, the oxidative hair treatment agent according to the invention comprises at least one oxidizing agent, which is selected from the inorganic salts of a peroxysulfuric acid, and mixtures thereof.

Peroxysulfuric acids are understood to mean peroxydisulfuric acid and peroxymonosulfuric acid (Caro's acid).

The at least one inorganic salt of a peroxysulfuric acid is preferably selected from ammonium peroxydisulfate, ammonium peroxymonosulfate, alkali metal peroxydisulfates, alkali metal peroxymonosulfates, and alkali metal hydrogen peroxymonosulfates. Particular preference is given to potassium peroxydisulfate, sodium peroxydisulfate, ammonium peroxydisulfate, and potassium hydrogen peroxymonosulfate, and mixtures thereof. Moreover, it has proven to be particularly preferred while conducting the work of the present invention for the oxidative hair treatment agent according to the invention to contain at least two different peroxydisulfates. Preferred peroxydisulfate salts are potassium peroxydisulfate-ammonium peroxydisulfate mixtures, potassium peroxydisulfate-ammonium peroxydisulfate-sodium peroxydisulfate mixtures, and potassium peroxydisulfate-sodium peroxydisulfate mixtures. Particular preference is given to mixtures of potassium peroxydisulfate and ammonium peroxydisulfate, and exceptionally preferably to mixtures of potassium peroxydisulfate and ammonium peroxydisulfate, with an excess of potassium peroxydisulfate, which are free of sodium peroxydisulfate. Mixtures of potassium peroxydisulfate (KPS) and ammonium peroxydisulfate (APS) that are particularly preferred according to the invention and are free of sodium peroxydisulfate are those having a KPS/APS weight ratio in the range of 4:1 to 2:1, preferably 3.5:1 to 2.5:1, and particularly preferably 3.2:1 to 2.8:1.

Preferred oxidative hair treatment agents according to the invention contain at least one oxidizing agent, which is selected from inorganic salts of a peroxysulfuric acid and mixtures thereof, in a total amount of 5 to 85 wt %, preferably 10 to 70 wt %, particularly preferably 17 to 55 wt %, and exceptionally preferably 22 to 45 wt %, in each case relative to the weight of the oxidative hair treatment agent.

The oxidative hair treatment agent according to the invention contains, as a second essential component, at least one complexing agent b) selected from
b) i) complexing agents of the general formula (I),

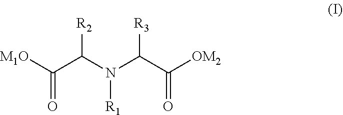

where
R1 represents a hydrogen atom, a C₁-C₆ alkyl group, a hydroxy-C₂-C₆ alkyl group, a carboxy-C₁-C₆ alkyl group, or a physiologically tolerated salt thereof,
R2, R3, independently of one another, represent a hydrogen atom, a C₁-C₆ alkyl group, a hydroxy-C₂-C₆ alkyl group, a carboxy-C₁-C₆ alkyl group, or a physiologically tolerated salt thereof, wherein R2 and R3 do not simultaneously represent a hydrogen atom and do not simultaneously represent a carboxy-C₁-C₆ alkyl group,
M1, M2, independently of one another, represent a hydrogen atom or an alkali metal ion—preferably sodium, potassium, or an ammonium ion (NH₄⁺)—and
b) ii) complexing agents of the general formula (II)

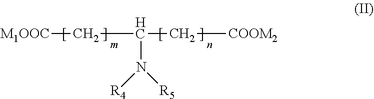

where
R4, R5, independently of one another, represent a carboxy-C₁-C₆ alkyl group or a physiologically tolerated salt thereof, a hydrogen atom, a C₁-C₆ alkyl group, or a hydroxy-C₂-C₆ alkyl group, and
m represents an integer from 0 to 6, and
n represents an integer from 0 to 6, and

M1, M2, independently of one another, represent a hydrogen atom or an alkali metal ion—preferably sodium, potassium, or an ammonium ion (NH₄⁺),
b) iii) ethylenediamine-N,N′-disuccinic acid or at least one alkali metal salt or ammonium salt of this acid.

In a further embodiment of the invention, the oxidative hair treatment agent according to the invention contains, as a second essential component, at least one complexing agent b) i) selected from complexing agents of the general formula (I),

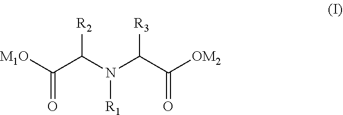

where
R1 represents a hydrogen atom, a C₁-C₆ alkyl group, a hydroxy-C₂-C₆ alkyl group, a carboxy-C₁-C₆ alkyl group, or a physiologically tolerated salt thereof,
R2, R3, independently of one another, represent a hydrogen atom, a C₁-C₆ alkyl group, a hydroxy-C₂-C₆ alkyl group, a carboxy-C₁-C₆ alkyl group, or a physiologically tolerated salt thereof, wherein R2 and R3 do not represent a hydrogen atom and do not simultaneously represent a carboxy-C₁-C₆ alkyl group, and
M1, M2, independently of one another, represent a hydrogen atom or an alkali metal ion—preferably sodium, potassium, or an ammonium ion (NH₄⁺).

Examples of the substituents R1, R2, and R3 stated in formula (I), which are particularly suitable according to the invention, are listed below by way of example. Examples of C₁-C₆ alkyl functional groups are —CH₃, —CH₂CH₃, —CH₂CH₂CH₃, —CH(CH₃)₂, —CH₂CH(CH₃)₂, —CH(CH₃)CH₂CH₃, —C(CH₃)₃, —(CH₂)₄CH₃, —(CH₂)₅CH₃. Particularly preferred C₁-C₆ alkyl functional groups are methyl and ethyl; an exceptionally preferred C₁-C₆ alkyl functional group is methyl. Examples of C₁-C₆ hydroxyalkyl groups are —CH₂OH, —CH₂CH₂OH, —CH₂CH₂CH₂OH, —CH₂CH(OH)CH₃, —CH₂CH₂CH₂CH₂OH, where —CH₂CH₂OH is preferred.

Examples of carboxy-C₁-C₆ alkyl groups are HOOC—CH₂—, HOOC—CH₂—CH₂—, HOOC—CH₂—CH₂—CH₂—, HOOC—CH₂—CH₂—CH₂—CH₂—, wherein the group HOOC—CH₂— is exceptionally preferred according to the invention.

In the complexing agents of the general formula (I), the functional group R1 represents a hydrogen atom, a C₁-C₆ alkyl group, a hydroxy-C₂-C₆ alkyl group, a carboxy-C₁-C₆ alkyl group, or a physiologically tolerated salt of a carboxy-C₁-C₆ alkyl group.

Physiologically acceptable salts are understood to mean the salts which can be used in cosmetics under physiological conditions without disadvantageous effect. Examples of a physiologically tolerated salt of a carboxy-C₁-C₆ alkyl group are, for example, the sodium salt, the potassium salt, and the ammonium salt of the carboxy-C₁-C₆ alkyl group.

A particularly strong improvement with regard to the reduced heat development and the bleaching power could be observed with agents according to the invention that contained at least one complexing agent (a) of the general formula (I), in which the functional group R1 represents a carboxy-C₁-C₆ alkyl group or a physiologically tolerated salt thereof, preferably a carboxymethyl group or a physiologically tolerated salt thereof, and particularly preferably the sodium salt of a carboxymethyl group.

In the context of a further particularly preferred embodiment, an agent according to the invention is characterized in that it contains at least one complexing agent (b) i) of general formula (I), where
R1 represents a carboxy-C₁-C₆ alkyl group or a physiologically tolerated salt thereof, preferably a carboxymethyl group or a physiologically tolerated salt thereof, and particularly preferably the sodium salt of a carboxymethyl group.

In the complexing agents of the general formula (I), the functional groups R2 and R3 represent, independently of one another, a hydrogen atom, a C₁-C₆ alkyl group, a hydroxy-C₂-C₆ alkyl group, a carboxy-C₁-C₆ alkyl group, or a physiologically tolerated salt thereof, wherein R2 and R3 do not simultaneously represent one hydrogen atom and do not simultaneously represent one carboxy-C₁-C₆ alkyl group.

In bleaching tests, particularly good results were also obtained with regard to the lightening power and the reduced heat development when at least one complexing agent (b) i) of the formula (I), in which one of the functional groups R2 or R3 represents a hydrogen atom and the other of the functional groups R2 or R3 represents a methyl group, was used in the agent according to the invention.

In the context of a particularly preferred embodiment, an agent according to the invention is characterized in that it contains at least one complexing agent (b) i) of the general formula (I), in which one of the functional groups R2 or R3 represents a hydrogen atom, and the other of the functional groups R2 or R3 represents a methyl group.

The best results regarding the lightening power and the reduced heat development were obtained with complexing agents (b) i) of the formula (I), in which
R1 represents a carboxymethyl group or a physiologically tolerated salt thereof,
R2 represents a methyl group, and
R3 represents a hydrogen atom.

In the context of a further particularly preferred embodiment, an agent according to the invention is characterized in that it contains at least one complexing agent (b) i) of general formula (I), where
R1 represents a carboxymethyl group or an alkali metal or ammonium salt thereof,
R2 represents a methyl group, and
R3 represents a hydrogen atom.

An explicitly very particularly preferred complexing agent (b) i) of this embodiment is N,N-bis(carboxymethyl)-L-alanine, which can alternatively also be referred to as 2-methyl-2′,2″,2″-nitrotriacetic acid, and is abbreviated as the substance MGDA (methyl glycine diacetic acid). MGDA has the CAS number 29578-05-0. MGDA has the formula (I-a). The alkali metal salts and ammonium salts of MGDA are also in accordance with the invention. The trisodium methyl glycine diacetate is particularly preferred.

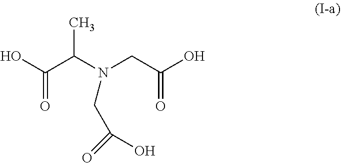

MGDA has a stereogenic center. Both the D-form and the L-form and a mixture of the D- and L-forms are in accordance with the invention.

According to the invention, particularly preferred oxidative hair treatment agents are characterized in that the at least one complexing agent b) i), which is selected from complexing agents (b) i) of formula (I) or an alkali metal salt or ammonium salt of this acid, is present in a total amount of 0.1 to 5.0 wt %, preferably 0.5 to 3.0 wt %, particularly preferably 1.0 to 2.0 wt %, and exceptionally preferably 1.4-1.6 wt %, in each case relative to the weight of the oxidative hair treatment agent according to the invention.

Other particularly preferred oxidative hair treatment agents according to the invention are characterized in that the at least one complexing agent b) i), which is selected from methyl glycine diacetic acid and its alkali metal salts or ammonium salts, is present in a total amount of 0.1 to 5.0 wt %, preferably 0.5 to 3.0 wt %, particularly preferably 1.0 to 2.0 wt %, and exceptionally preferably 1.4-1.6 wt %, in each case relative to the weight of the oxidative hair treatment agent.

Other particularly preferred oxidative hair treatment agents according to the invention are characterized in that the at least one complexing agent b) i), which is selected from the sodium salts of the methyl glycine diacetic acid, is present in a total amount of 0.1 to 5.0 wt %, preferably 0.5 to 3.0 wt %, particularly preferably 1.0 to 2.0 wt %, and exceptionally preferably 1.4-1.6 wt %, in each case relative to the weight of the oxidative hair treatment agent.

Other particularly preferred oxidative hair treatment agents according to the invention are characterized in that the trisodium salt of methyl glycine diacetic acid is present in an amount of 0.1 to 5.0 wt %, preferably 0.5 to 3.0 wt %, particularly preferably 1.0 to 2.0 wt %, and exceptionally preferably of 1.4 to 1.6 wt %, in each case relative to the weight of the oxidative hair treatment agent.

In a further embodiment of the invention, the oxidative hair treatment agent according to the invention contains, as a second essential component, at least one complexing agent b) ii) selected from complexing agents of the general formula (II),

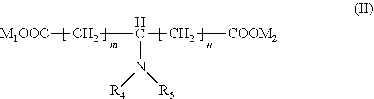

where
R4, R5, independently of one another, represent a carboxy-C₁-C₆ alkyl group or a physiologically tolerated salt thereof, a hydrogen atom, a C₁-C₆ alkyl group, or a hydroxy-C₂-C₆ alkyl group, and
m represents an integer from 0 to 6, and
n represents an integer from 0 to 6, and
M1, M2, independently of one another, represent a hydrogen atom or an alkali metal ion—preferably sodium or potassium, or an ammonium ion (NH₄⁺).

Examples of the substituents R4 and R5 stated in formula (II) are listed below by way of example:

Examples of C₁-C₆ alkyl functional groups are —CH₃, —CH₂CH₃, —CH₂CH₂CH₃, —CH(CH₃)₂, —CH₂CH(CH₃)₂, —CH(CH₃)CH₂CH₃, —C(CH₃)₃, —(CH₂)₄CH₃, —(CH₂)₅CH₃. Methyl and ethyl are particularly preferred alkyl functional groups. Examples of C₁-C₆ hydroxyalkyl groups are —CH₂OH, —CH₂CH₂OH, —CH₂CH₂CH₂OH, —CH₂CH(OH)CH₃, —CH₂CH₂CH₂CH₂OH, where —CH₂CH₂OH is preferred.

Examples of carboxy-C₁-C₆ alkyl groups are HOOC—CH₂—, HOOC—CH₂—CH₂—, HOOC—CH₂—CH₂—CH₂—, HOOC—CH₂—CH₂—CH₂—CH₂—, where the group HOOC—CH₂— is preferred.

In the complexing agents of the general formula (II), the functional groups R4 and R5 independently of one another represent a carboxy-C₁-C₆ alkyl group or a physiologically tolerated salt thereof, a hydrogen atom, a C₁-C₆ alkyl group, or a hydroxy-C₂-C₆ alkyl group.

Physiologically acceptable salts are understood to mean the salts which can be used in cosmetics under physiological conditions without disadvantageous effect. Examples of a physiologically acceptable salt of a carboxyl group are the sodium salt, the potassium salt, and the ammonium salt. Examples of a physiologically tolerated salt of a carboxy-C₁-C₆ alkyl group are, for example, the sodium salt, the potassium salt, and the ammonium salt of the carboxy-C₁-C₆ alkyl group.

A particularly strong reduction in the exotherm was observed when using agents that contained at least one complexing agent (b)ii) of general formula (I), where the functional groups R4 and R5, independently of one another, represent a carboxy-C₁-C₆ alkyl group or an alkali metal salt or ammonium salt thereof.

In the context of a further particularly preferred embodiment, an agent according to the invention is characterized in that it contains at least one complexing agent (b) of general formula (II), where
R4, R5 independently of one another, represent a carboxy-C₁-C₆ alkyl group or an alkali metal salt or ammonium salt thereof.

Very particularly preferably, R4 and R5, independently of one another, represent a carboxy-C₁-C₆ alkyl group or an alkali metal salt or ammonium salt thereof. Explicitly, very particularly preferably, R4 and R5, independently of one another, represent a carboxymethyl group or an alkali metal salt or ammonium salt thereof.

In the context of an explicitly very particularly preferred embodiment, an agent according to the invention is characterized in that it contains at least one complexing agent (b)ii) of general formula (II), where
R4, R5, independently of one another, represent a carboxymethyl group or an alkali metal salt or ammonium salt thereof.

In the context of an explicitly very particularly preferred embodiment, an agent according to the invention is characterized in that it contains at least one complexing agent (b)ii) of general formula (II), where
R4, R5 both represent a carboxymethyl group or an alkali metal salt or an ammonium salt thereof.

In the complexing agents of general formula (II), the index number m represents an integer from 0 to 6. The index number m indicates the number of methylene groups (i.e., —CH₂ groups) which are between the structural unit —COOM₁ and the carbon atom bearing the grouping —NR₁R₂. Particularly good results were obtained when complexing agents of formula (II) were used in which m represented the number 0 or 1, and very particularly preferably the number 0.

In the complexing agents of general formula (II), the index number n represents an integer from 0 to 6. The index number n indicates the number of methylene groups (i.e., —CH₂ groups) which are located between the carbon atom bearing the grouping —NR1R2 and the grouping —COOM₂. In this context, particularly good results were obtained when complexing agents of formula (II) were used in which n represented the number 0, 1, or 2, and very particularly preferably the number 2.

In the context of a further particularly preferred embodiment, an agent according to the invention is characterized in that it contains at least one complexing agent (b)i) of general formula (II), where
m represents the number 0 or 1, and preferably the number 0, and/or
n represents the number 0, 1, or 2, and preferably the number 2.

In the context of a further particularly preferred embodiment, an agent according to the invention is characterized in that it contains at least one complexing agent (b)ii) of general formula (II), where
m represents the number 0, and
n represents the number 2.

If R4 and R5, independently of one another, represent a carboxymethyl group or an alkali metal salt or ammonium salt thereof, m represents the number 0, and n represents the number 2, then the complexing agent (a) has the general structural formula (IIa).

In the context of a further particularly preferred embodiment, an agent according to the invention is characterized in that it contains at least one complexing agent (b)ii) of general formula (IIa),

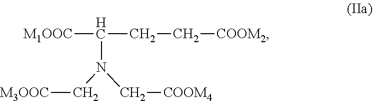

where
M1, M2, independently of one another, represent a hydrogen atom or an alkali metal ion—preferably sodium or potassium, or an ammonium ion (NH₄⁺)—and
M3, M4, independently of one another, represent a hydrogen atom or an alkali metal ion—preferably sodium or potassium, or an ammonium ion (NH₄⁺).

The preferred compound of formula (IIa) is called glutamic acid N,N-diacetic acid (GLDA).

To achieve the object according to the invention, it has been found to be very particularly advantageous if M1, M2, M3, and M4, independently of one another, represent a sodium cation (Na⁺) or a potassium cation (K⁺).

In the context of a particularly preferred embodiment, an agent according to the invention is characterized in that it contains at least one complexing agent (b)ii) of general formula (IIa),

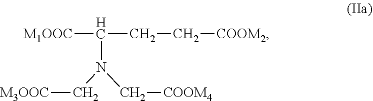

where
M1, M2, independently of one another, represent sodium or potassium, and
M3, M4, independently of one another, represent sodium or potassium.

An explicitly very particularly preferred complexing agent (b)ii) of this embodiment is tetrasodium N,N-bis(carboxylatomethyl)-L-glutamate. Tetrasodium N,N-bis(carboxylatomethyl)-L-glutamate falls under the formula (IIa), where M1, M2, M3, and M4 represent a sodium cation.

Tetrasodium N,N-bis(carboxylatomethyl)-L-glutamate can alternatively also be referred to as glutamic acid N,N diacetic acid tetrasodium salt. The L-form carries the name, L-tetrasodium N,N-bis(carboxylatomethyl)-L-glutamate or (S)-glutamic acid N,N diacetic acid tetrasodium salt, N,N-bis(carboxymethyl)-L-glutamic acid tetrasodium salt, or GLDA-Na₄ for short and has the CAS number 51981-21-6. The INCI name of this compound is Tetrasodium Glutamate Diacetate.

Glutamic acid N,N-diacetic acid (GLDA) has a stereogenic center. Both the (S)-form and the (R)-form (or both the L-form and the D-form) are according to the invention.

According to the invention, particularly preferred oxidative hair treatment agents are characterized in that the at least one complexing agent b) ii), which is selected from complexing agents (b) ii) of formula (II) or an alkali metal salt or ammonium salt of this complexing agent, is present in a total amount of 0.1 to 5.0 wt %, preferably 0.5 to 3.0 wt %, particularly preferably 1.0 to 2.0 wt %, and exceptionally preferably 1.4-1.6 wt %, in each case relative to the weight of the oxidative hair treatment agent according to the invention.

Other particularly preferred oxidative hair treatment agents according to the invention are characterized in that the at least one complexing agent b) ii), which is selected from glutamic acid N,N-diacetic acid and its alkali metal salts or ammonium salts, is present in a total amount of 0.1 to 5.0 wt %, preferably from 0.5 to 3.0 wt %, particularly preferably from 1.0 to 2.0 wt %, and exceptionally preferably from 1.4 to 1.6 wt %, in each case relative to the weight of the oxidative hair treatment agent.

Other particularly preferred oxidative hair treatment agents according to the invention are characterized in that the at least one complexing agent b) ii), which is selected from the sodium salts of glutamic acid N,N-diacetic acid, is present in a total amount of 0.1 to 5.0 wt %, preferably 0.5 to 3.0 wt %, particularly preferably 1.0 to 2.0 wt %, and exceptionally preferably 1.4-1.6 wt %, in each case relative to the weight of the oxidative hair treatment agent.

Other particularly preferred oxidative hair treatment agents according to the invention are characterized in that the tetrasodium salt of glutamic acid N,N-diacetic acid is present in an amount of 0.1 to 5.0 wt %, preferably 0.5 to 3.0 wt %, particularly preferably 1.0 to 2.0 wt %, and exceptionally preferably 1.4-1.6 wt %, in each case relative to the weight of the oxidative hair treatment agent.

Other preferred oxidative hair treatment agents according to the invention are characterized in that the at least one salt of ethylenediamine-N,N′-disuccinic acid is selected from sodium salts, potassium salts, and ammonium salts, and from mixtures of these salts.

Oxidative hair treatment agents that are particularly preferred according to the invention are characterized in that complexing agent b) iii) is selected from the monosodium salt, disodium salt, trisodium salt, and tetrasodium salt of ethylenediamine-N,N′-disuccinic acid, and mixtures thereof, wherein the trisodium salt and the tetrasodium salt of ethylenediamine-N, N′-disuccinic acid are preferred, and the trisodium salt of ethylenediamine-N,N′-disuccinic acid is particularly preferred.

Other especially preferred oxidative hair treatment agents according to the invention are characterized in that the at least one complexing agent b) iii), which is selected from ethylenediamine-N,N′-disuccinic acid or an alkali metal salt or ammonium salt of this acid, is present in a total amount of 0.1 to 5.0 wt %, preferably 0.5 to 3.0 wt %, particularly preferably 1.0 to 2.0 wt %, and exceptionally preferably 1.4-1.6 wt %, in each case relative to the weight of the oxidative hair treatment agent according to the invention.

Other particularly preferred oxidative hair treatment agents according to the invention are characterized in that the at least one complexing agent b) iii), which is selected from the sodium salts of ethylenediamine-N,N′-disuccinic acid, is present in a total amount of 0.1 to 5.0 wt %, preferably 0.5 to 3.0 wt %, particularly preferably 1.0 to 2.0 wt %, and exceptionally preferably 1.4 to 1.6 wt %, in each case relative to the weight of the oxidative hair treatment agent.

Other particularly preferred oxidative hair treatment agents according to the invention are characterized in that the trisodium salt of ethylenediamine-N,N′-disuccinic acid is present in an amount of 0.1 to 5.0 wt %, preferably 0.5 to 3.0 wt %, particularly preferably 1.0 to 2.0 wt %, and exceptionally preferably 1.4 to 1.6 wt %, in each case relative to the weight of the oxidative hair treatment agent.

The oxidative hair treatment agent according to the invention contains, as a third essential component, at least one amino acid selected from alpha-amino acids, beta-amino acids, and the salts of an alpha-amino acid or beta-amino acid, and mixtures of these substances.

An amino acid is understood to mean a chemical compound having an amino group and a carboxylic acid group. The class of amino acids includes organic compounds which contain at least one amino group (—NH₂ or substituted —NR₂) and a carboxy group (—COOH) as functional groups, i.e., have structural features of amines and carboxylic acids. They can be chemically distinguished according to the position of their amino group relative to the carboxy group. If the amino group is at the Cₐₗₚₕₐ atom immediately adjacent to the terminal carboxy group, it is said to be in the alpha position (α-position) and the term, alpha-amino acids (α-amino acids), is used. If the amino group is at the C_{beta} atom, as seen from the terminal carboxy group, it is said to be in the beta position (β-position) and the term, beta-amino acids (β-amino acids), is used. Alpha-amino acids and beta-amino acids having a total number of C atoms of C2-C20, more preferably of C2-C15, and particularly preferably of C2-C10, are preferably present.

In the context of a further particularly preferred embodiment, an agent according to the invention is characterized in that it contains at least one amino acid (c) from the group consisting of arginine, serine, histidine, lysine, asparagine, glutamine, cysteine, methionine, tryptophan, alanine, aspartic acid, glutamic acid, glycine, isoleucine, leucine, phenylalanine, proline, threonine, tyrosine, valine, and taurine, the salts of the aforementioned amino acids, and mixtures of these substances.

Particularly preferred amino acids (c) according to the invention are selected from the group consisting of arginine, serine, the salts of the aforementioned amino acids, and mixtures of these substances.

Compositions that are exceptionally preferred according to the invention are characterized in that they contain, in most cases, an amino acid (c) selected from the group consisting of arginine, serine, the salts of the aforementioned amino acids, and mixtures of these substances.

Chiral amino acids have a stereogenic center and can occur in mirror image forms. For example, arginine occurs in the form of L-arginine and D-arginine. Both the L-form of an amino acid and its D-form and the mixtures thereof are encompassed by the present invention. In the context of the present invention, both possible enantiomers can therefore equally be used as a specific compound or else mixtures thereof, and in particular as racemates. However, it is particularly advantageous to use the naturally occurring isomer form, usually in the L configuration.

Accordingly, a particularly preferred agent according to the invention is characterized in that it contains at least one amino acid (c) from the group consisting of L-arginine, L-serine, L-histidine, L-lysine, L-asparagine, L-glutamine, L-cysteine, L-methionine, L-tryptophan, L-alanine, L-aspartic acid, L-glutamic acid, glycine, L-isoleucine, L-leucine, L-phenylalanine, L-proline, L-threonine, L-tyrosine, and L-valine, and taurine, the salts of the aforementioned amino acids, and mixtures of these substances.

Other preferred oxidative hair treatment agents according to the invention are characterized in that the at least one alpha-amino acid or beta-amino acid c) and/or its salt is present in a total amount, converted to the mass of free amino acid, of 0.1 to 3.0 wt %, preferably 0.2 to 1.5 wt %, particularly preferably 0.3 to 0.6 wt %, and exceptionally preferably 0.4 to 0.5 wt %, in each case relative to the weight of the oxidative hair treatment agent.

Exceptionally preferred oxidative hair treatment agents according to the invention contain at least one alpha-amino acid c), selected from arginine, serine, or at least one salt of these amino acids, in a total amount, converted to the mass of free amino acid, of 0.1 to 3.0 wt %, preferably 0.2 to 1.5 wt %, particularly preferably 0.3 to 0.6 wt %, and exceptionally preferably 0.4 to 0.5 wt %, in each case relative to the weight of the oxidative hair treatment agent.

Exceptionally preferred oxidative hair treatment agents according to the invention contain at least one amino acid, selected from L-arginine, L-serine, or at least one salt of these amino acids, in a total amount, converted to the mass of free amino acid, of 0.1 to 3.0 wt %, preferably 0.2 to 1.5 wt %, particularly preferably 0.3 to 0.6 wt %, and exceptionally preferably 0.4 to 0.5 wt %, in each case relative to the weight of the oxidative hair treatment agent.

The oxidative hair treatment agent according to the invention contains Polyquaternium-4 as the fourth essential component. Polyquaternium-4 is hydroxyethyl cellulose quaternized with diallyldimethylammonium chloride (INCI: Polyquaternium-4). Surprisingly, it was found that Polyquaternium-4 can influence the swelling behavior of the oxidative hair treatment agent according to the invention very favorably after mixing with an aqueous hydrogen peroxide preparation. Polyquaternium-4 is particularly preferably present in a total amount of 0.01 to 3.0 wt %, preferably 0.1 to 1.0 wt %, particularly preferably 0.1 to 0.7 wt %, and exceptionally preferably 0.3 to 0.5 wt %, in each case relative to the weight of the oxidative hair treatment agent.

For the melanin-degrading effect of the hydrogen peroxide and the bleaching effect on the keratin fibers, it is advantageous if the mixture to be used formed of hydrogen peroxide solution and hair treatment agent containing persalt has an alkaline pH value, which lies in the range of 8.0 to 11.5, preferably in the range of 8.5 to 11.0, particularly preferably in the range of 9.0 to 10.5, and exceptionally preferably in the range of 9.5 to 10.3, measured in each case at 20° C.

In order to set an alkaline pH value of the lightening mixture to be used, oxidative hair treatment agents preferred according to the invention can contain, in addition to the at least one persalt, at least one powdery alkalizing agent in such a total amount that the mixture to be used has the desired alkaline pH value. Alternatively, a third composition can be added to the mixture to be used of oxidative hair treatment agent according to the invention and hydrogen peroxide solution, which third composition contains one or more alkalizing agents in such an amount that the mixture to be used has an alkaline pH value, which lies in the range of 8.0 to 11.5, preferably in the range of 8.5 to 11.0, particularly preferably 9.0 to 10.5, and exceptionally preferably 9.5 to 10.3, measured in each case at 20° C.

Preferred oxidative hair treatment agents according to the invention are therefore characterized in that at least one inorganic alkalizing agent that is solid at 20° C. and 1,013 mbar is present. Particularly preferred oxidative hair treatment agents according to the invention additionally contain at least one inorganic alkalizing agent that is solid at 20° C. and 1,013 mbar, which is preferably present in a total amount of 4 to 70 wt %, preferably 10 to 65 wt %, particularly preferably 15 to 60 wt %, and exceptionally preferably 20 to 55 wt %, in each case relative to the weight of the oxidative hair treatment agent.

Other preferred oxidative hair treatment agents according to the invention contain at least one inorganic alkalizing agent that is solid at 20° C. and 1,013 mbar, including, in a total amount of 0.1 to 50 wt %, preferably 4 to 30 wt %, and particularly preferably 15 to 25 wt %, in each case relative to the weight of the oxidative hair treatment agent, at least one sodium silicate or sodium metasilicate having a molar SiO₂/Na₂O ratio of ≥2, and preferably 2.5-3.5.

Besides the at least one sodium silicate or sodium metasilicate having a molar SiO₂/Na₂O ratio of ≥2, and preferably 2.5-3.5, in a total amount of 0.1 to 50 wt %, preferably 4 to 30 wt %, and particularly preferably 15 to 25 wt %, in each case relative to the weight of the oxidative hair treatment agent, as an optional alkalizing agent, other particularly preferred alkalizing agents according to the invention that are solid at 20° C. and 1,013 mbar are selected from alkaline earth metal silicates, alkaline earth metal hydroxide carbonates, alkaline earth metal carbonates, alkaline earth metal metasilicates, alkali metal hydroxides, alkaline earth metal hydroxides, alkali (alkaline earth) metal phosphates, and alkali (alkaline earth) metal hydrogen phosphates, and mixtures of these substances. Particularly preferred inorganic alkalizing agents according to the invention that are solid at 20° C. and 1,013 mbar, in addition to the at least one obligatory sodium silicate or sodium metasilicate, in each case having a molar SiO₂/Na₂O ratio of ≥2, and preferably 2.5 to 3.5, are selected from magnesium hydroxide carbonates, and mixtures of these alkalizing agents. Preferred magnesium hydroxide carbonates according to the invention are those of formula MgCO₃·Mg(OH)₂·2 H₂O and those of formula MgCO₃·Mg(OH)₂. Magnesium hydroxide carbonate of formula MgCO₃·Mg(OH)₂ is particularly preferred according to the invention.

Particularly preferred oxidative hair treatment agents according to the invention contain, in each case relative to the total weight thereof, 0.1 to 50 wt %, preferably 4 to 30 wt %, and particularly preferably 15 to 25 wt % sodium silicates having a molar SiO₂/Na₂O ratio of ≥2, and preferably 2.5 to 3.5, and 2 to 40 wt %, preferably 5 to 35 wt %, and particularly preferably 10 to 32 wt % magnesium hydroxide carbonate as inorganic alkalizing agents that are solid at 20° C. and 1,013 mbar.

The oxidative hair treatment agents according to the invention are anhydrous, which in the sense of the present invention means that they contain, in each case relative to the weight thereof, 0 to 10 wt %, preferably 0.1 to 8 wt %, and particularly preferably 0.5 to 5 wt % water.

These values relate to the content of free water. A content of molecularly-bound water or water of crystallization that individual powder components may comprise is not taken into account. The water content can be determined, for example, based upon ISO 4317 (Version 2011-12) by means of Karl Fischer titration.

The oxidative hair treatment agents according to the invention are further characterized in that no polymer is contained that comprises acrylic acid, acrylic acid amides, acrylic esters, methacrylic acid, methacrylic acid amides, and/or methacrylic esters as monomers. Such polymers can adversely affect the heat development in the ready-to-use bleaching agent.

For safety reasons, the oxidative hair treatment agents, which are packaged as a bleaching powder, can be formulated in such a way that, when used—in particular, before or while being mixed with the liquid hydrogen peroxide solution—they do not become dusty, or become as little dusty as possible. Suitable means for dust reduction are in particular oils mixed with the dust-producing components of the bleaching powder, wherein the total amount of oils is 0.2 to 20 wt %, relative to the weight of the oxidative hair treatment agent according to the invention, which is present as a bleaching powder.

If more than 20 wt % oil is used, a paste is formed with the anhydrous powdery components. Oxidative hair treatment agents according to the invention that are present as bleaching paste contain, in each case relative to their weight, at least one oil in a total amount of 20.1 to 80 wt %, and preferably 35 to 65 wt %.

The application properties of the oxidative hair treatment agent and of the entire ready-to-use lightening agent can be optimized by skillful selection of the oils. The selection of oils should also take into account the consumer's desire for products that contain ingredients containing as few synthetically produced ingredients as possible, but, rather, naturally sourced ingredients. Oils of mineral origin, such as mineral oils and paraffins, for example, have good application properties and are inexpensive. For the sustainability of the entire product, it is preferable to use oils from renewable raw material sources rather than oils of mineral origin. Surprisingly, it has been found that certain oils can influence the swelling behavior of the oxidative hair treatment agent according to the invention very favorably after mixing with an aqueous hydrogen peroxide preparation.

Preferred oxidative hair treatment agents according to the invention contain, relative to their weight, at least one oil in a total amount of 0.2 to 80 wt %, preferably 1 to 70 wt %, particularly preferably 4 to 60 wt %, and exceptionally preferably 5 to 20 wt %.

According to the invention, the at least one oil is preferably selected from mineral oils, branched fatty alcohols having 8-24 carbon atoms, dialkyl ethers having 6 to 18 C atoms in the alkyl groups, and mixtures of the aforementioned substances.

Preferred branched fatty alcohols according to the invention having 8-24 carbon atoms are 2-octyl-1-dodecanol, 2-hexyl-1-decanol, 2-ethyl-1-hexanol, and isostearyl alcohol, and mixtures thereof. 2-octyl-1-dodecanol is exceptionally preferred.

Preferred dialkyl ethers according to the invention having 6 to 18 C atoms in the alkyl groups are di-n-alkyl ethers having a total of 12 to 36 C atoms, and in particular 16 to 24 C atoms in the molecule, such as, for example, di-n-octyl ether, di-n-decyl ether, di-n-nonyl ether, di-n-undecyl ether, di-n-dodecyl ether, di-n-octadecyl ether, n-hexyl-n-octyl ether, n-octyl-n-decyl ether, n-decyl-n-undecyl ether, n-undecyl-n-dodecyl ether, and n-hexyl-n-undecyl ether. Likewise preferred dialkyl ethers are those with branched alkyl groups in each case with 6 to 18 C atoms, and in particular with alkyl groups that are substituted in the 2-position with an ethyl group. Preferred branched-chain dialkyl ethers having 6 to 18 C atoms in the alkyl group are selected from di-(2-ethylhexyl) ether and di-(2-ethyl-decyl) ether. Particular preference is given to di-n-octyl ether (INCI: Dicaprylyl ether), which is commercially available, e.g., under the name, Cetiol® OE, from BASF.

Preferred oxidative hair treatment agents according to the invention are characterized in that they contain, relative to their weight, at least one oil selected from mineral oils, branched fatty alcohols having 8-24 carbon atoms, dialkyl ethers having 6 to 18 C atoms in the alkyl groups, and mixtures of the aforementioned substances, in a total amount of 0.2 to 80 wt %, preferably 1 to 70 wt %, particularly preferably 4 to 60 wt %, and exceptionally preferably 5 to 20 wt %.

Exceptionally preferred oxidative hair treatment agents according to the invention are characterized in that they contain, relative to their weight, at least one oil selected from mineral oils, branched fatty alcohols having 8-24 carbon atoms, dialkyl ethers having 6 to 18 C atoms in the alkyl groups, and mixtures of the aforementioned substances, in a total amount of 0.2 to 10 wt %, preferably 1 to 7 wt %, particularly preferably 4 to 6 wt %, and exceptionally preferably 5 to 5.5 wt %.

Other exceptionally preferred inventive oxidative hair treatment agents according to the invention are characterized in that they contain, relative to their weight, at least one oil selected from mineral oils and 2-octyl-1-dodecanol and mixtures of the aforementioned substances, in a total amount of 0.2 to 10 wt %, preferably 1 to 7 wt %, particularly preferably 4 to 6 wt %, and exceptionally preferably 5 to 5.5 wt %.

Other exceptionally preferred oxidative hair treatment agents according to the invention are characterized in that they contain, relative to their weight, at least one oil selected from mineral oils and di-n-octyl ether and mixtures of the aforementioned substances, in a total amount of 0.2 to 10 wt %, preferably 1 to 7 wt %, particularly preferably 4 to 6 wt %, and exceptionally preferably 5 to 5.5 wt %.

Furthermore, silicone oils are also suitable oils according to the invention. Ester oils, e.g., benzoic acid esters of linear or branched C8-22 alkanols, such as benzoic acid C12-C15 alkyl esters, and triglycerides (i.e., triple esters of glycerol) of linear or branched, saturated or unsaturated, optionally hydroxylated C8-30 fatty acids, preferably saturated triglyceride oils, and in particular capric/caprylic triglycerides, and natural oils, e.g., amaranth seed oil, apricot kernel oil, argan oil, avocado oil, babassu oil, cottonseed oil, borage seed oil, safflower oil, peanut oil, pomegranate seed oil, grapefruit seed oil, hemp oil, hazelnut oil, elderberry seed oil, currant seed oil, jojoba oil, linseed oil, macadamia nut oil, corn oil, almond oil, marula oil, evening primrose oil, olive oil, palm oil, palm kernel oil, Brazil nut oil, pecan oil, peach kernel oil, canola oil, castor oil, sea buckthorn pulp oil, sea buckthorn seed oil, sesame oil, soybean oil, sunflower oil, grapeseed oil, walnut oil, wild rose oil, wheat germ oil, and the liquid components of coconut oil, and the dicarboxylic ester of linear or branched C₂-C₁₀ alkanols, such as diisopropyl adipate, di-n-butyl adipate, di-(2-ethylhexyl) adipate, dioctyl adipate, diethyl/di-n-butyl/dioctyl sebacate, diisopropyl sebacate, dioctyl malate, dioctyl maleate, dicaprylyl maleate, diisooctyl succinate, di-2-ethylhexyl succinate, and di-(2-hexyldecyl) succinate, and esters of linear or branched, saturated or unsaturated fatty alcohols having 2-30 carbon atoms having linear or branched, saturated or unsaturated fatty acids having 2-30 carbon atoms, which may be hydroxylated, and preferably selected from preferably 2-hexyldecyl stearate, 2-hexyldecyl laurate, isodecyl neopentanoate, isononyl isononanoate, 2-ethylhexyl palmitate and 2-ethylhexyl stearate, isopropyl myristate, isopropyl palmitate, isopropyl stearate, isopropyl isostearate, isopropyl oleate, isooctyl stearate, isononyl stearate, isocetyl stearate, isononyl isononanoate, isotridecyl isononanoate, cetearyl isononanoate, 2-ethylhexyl laurate, 2-ethylhexyl isostearate, 2-ethylhexyl cocoate, 2-octyldodecyl palmitate, butyloctanoic acid 2-butyl octanoate, diisotridecyl acetate, n-butyl stearate, n-hexyl laurate, n-decyl oleate, oleyl oleate, oleyl erucate, erucyl oleate, erucylerucate, ethylene glycol dioleate, and ethylene glycol dipalmitate, and C₈-C₂₂ fatty alcohol esters of monovalent or polyvalent C₂-C₇ hydroxycarboxylic acids, and in particular the esters of glycolic acid, lactic acid, malic acid, tartaric acid, citric acid, and salicylic acid, e.g., C₁₂-C₁₅ alkyl lactate, and the symmetrical, asymmetrical, or cyclic esters of carbonic acid having C₃₋₂₂ alkanols, C₃₋₂₂ alkanediols, or C₃₋₂₂ alkanetriols, e.g., dicaprylyl carbonate, can likewise be used as oil according to the invention, but are less preferred due to the possible risk of hydrolysis or should only be present in small amounts of 0.1 to a maximum of 2 wt %, relative to the oxidative hair treatment agent according to the invention.

Surprisingly, it has been found that an alkali metal salt or alkaline earth metal salt of a C12-C24 monocarboxylic acid, which may be saturated or unsaturated, linear or branched, can influence the swelling behavior of the oxidative hair treatment agent according to the invention very favorably after mixing with an aqueous hydrogen peroxide preparation.

Other preferred oxidative hair treatment agents according to the invention are therefore characterized in that at least one alkali metal salt or alkaline earth metal salt of a C12-C24 monocarboxylic acid, which may be saturated or unsaturated, linear or branched, is present, wherein the C12-C24 monocarboxylic acid is preferably selected from lauric acid, myristic acid, palmitic acid, palmitoleic acid, stearic acid, isostearic acid, oleic acid, elaidic acid, petroselinic acid, arachidic acid, gadoleic acid, behenic acid, erucic acid, and brassidic acid, and mixtures thereof, wherein stearic acid and palmitic acid and mixtures thereof are particularly preferred, and wherein the potassium, magnesium, and calcium salts of the C12-C24 monocarboxylic acid are exceptionally preferred. In this connection, potassium stearate, potassium palmitate, magnesium stearate, magnesium palmitate, calcium stearate, calcium palmitate, and mixtures of these substances are exceptionally preferred, wherein potassium stearate is particularly preferred. Other exceptionally preferred oxidative hair treatment agents according to the invention are therefore characterized in that they contain, in each case relative to their weight, at least one alkali metal salt or alkaline earth metal salt of a C12-C24 monocarboxylic acid, which may be saturated or unsaturated, linear or branched, in a total amount of 0.05 to 1 wt %, preferably 0.2 to 0.6 wt %, and particularly preferably 0.3 to 0.5 wt %. Other exceptionally preferred oxidative hair treatment agents according to the invention are therefore characterized in that they contain, in each case relative to their weight, potassium stearate in a total amount of 0.05 to 1 wt %, preferably 0.2 to 0.6 wt %, and particularly preferably 0.3 to 0.5 wt %.

Surprisingly, it has been found that sodium hexametaphosphate can influence the swelling behavior of the oxidative hair treatment agent according to the invention very favorably after mixing with an aqueous hydrogen peroxide preparation. Other exceptionally preferred oxidative hair treatment agents according to the invention are therefore characterized in that they contain, in each case relative to their weight, 0.05 to 0.5 wt %, and preferably 0.2 to 0.4 wt % sodium hexametaphosphate.

Surprisingly, it has been found that kaolin can influence the swelling behavior of the oxidative hair treatment agent according to the invention very favorably after mixing with an aqueous hydrogen peroxide preparation. Other exceptionally preferred oxidative hair treatment agents according to the invention are therefore characterized in that they contain, in each case relative to their weight, 0.05 to 5 wt %, preferably 0.5 to 4 wt %, and particularly preferably 1 to 2 wt % kaolin.

In a further preferred embodiment, the oxidative hair treatment agent according to the invention further contains at least one dicarboxylic acid having 2 to 10 carbon atoms, and particularly preferably selected from succinic acid, oxalic acid, malonic acid, adipic acid, pimelic acid, suberic acid, azelaic acid, sebacic acid, malic acid, D-tartaric acid, L-tartaric acid, meso-tartaric acid, racemic acid, alpha-ketoglutaric acid, beta-ketoglutaric acid, oxaloacetic acid, maleic acid, and fumaric acid and/or at least one salt of these acids and mixtures of these compounds, wherein the at least one dicarboxylic acid having 2 to 10 carbon atoms is exceptionally preferably selected from succinic acid, malic acid, malonic acid, and maleic acid and salts thereof.

Preferred salts of dicarboxylic acids having 2 to 10 carbon atoms according to the invention are selected from the mono salts and di salts of the anions of succinic acid, oxalic acid, malonic acid, adipic acid, pimelic acid, suberic acid, azelaic acid, sebacic acid, malic acid, D-tartaric acid, L-tartaric acid, meso-tartaric acid, racemic acid, alpha-ketoglutaric acid, beta-ketoglutaric acid, oxaloacetic acid, maleic acid, and fumaric acid with alkali metal ions, alkaline earth metal ions, and the ions of basic amino acids, such as arginine, lysine, and histidine, and in particular with lithium, sodium, potassium, magnesium, and calcium ions and mixtures of these salts.

Succinic acid, which is particularly preferred according to the invention, has, at 1,013 mbar, a melting point in the range of 185-187° C., i.e., is thus a solid at 20° C. Suitable salts of succinic acid according to the invention are selected from the succinates and hydrogen succinates of alkali metal ions, alkaline earth metal ions, and the ions of basic amino acids, such as arginine, lysine, and histidine, and in particular the lithium, sodium, potassium, magnesium, and calcium ions, or the succinates and hydrogen succinates of basic amino acids, such as arginine, lysine, and/or histidine, such as arginine succinate, and mixtures of these salts. The specified salts of succinic acid can also contain bound water of crystallization, and in particular sodium succinate hexahydrate, which is particularly preferred in accordance with the invention.

Malic acid, which is particularly preferred in accordance with the invention, is optically active. For cost reasons, racemic DL-malic acid is preferred. Salts of malic acid suitable according to the invention are selected from the malates and hydrogen malates of alkali metal ions, alkaline earth metal ions, and the ions of basic amino acids, such as arginine, lysine, and histidine, and in particular of lithium, sodium, potassium, magnesium, and calcium ions and mixtures of these salts, and in particular disodium malate and dipotassium malate, but also calcium malate. The specified salts of malic acid suitable according to the invention can contain bound water of crystallization, and in particular disodium malate hemihydrate and disodium malate trihydrate.

Suitable salts of oxalic acid according to the invention are selected from the oxalates and hydrogen oxalates of alkali metal ions, alkaline earth metal ions, and the ions of basic amino acids, such as arginine, lysine, and histidine, and in particular of lithium, sodium, potassium, magnesium, and calcium ions, and mixtures of these salts.

Suitable salts of particularly preferable malonic acid according to the invention are selected from the malates and hydrogen malates of alkali metal ions, alkaline earth metal ions, and the ions of basic amino acids, such as arginine, lysine, and histidine, and in particular of lithium, sodium, potassium, magnesium, and calcium ions, and mixtures of these salts.

Suitable salts of adipic acid according to the invention are selected from the adipates and hydrogen adipates of alkali metal ions, alkaline earth metal ions, and the ions of basic amino acids, such as arginine, lysine, and histidine, and in particular of lithium, sodium, potassium, magnesium, and calcium ions, and mixtures of these salts.

Suitable salts of pimelic acid according to the invention are selected from the pimelates and hydrogen pimelates of alkali metal ions, alkaline earth metal ions, and the ions of basic amino acids, such as arginine, lysine, and histidine, and in particular of lithium, sodium, potassium, magnesium, and calcium ions, and mixtures of these salts.

Suitable salts of suberic acid according to the invention are selected from the suberates and hydrogen suberates alkali metal ions, alkaline earth metal ions, and the ions of basic amino acids, such as arginine, lysine, and histidine, and in particular of lithium, sodium, potassium, magnesium, and calcium ions, and mixtures of these salts.

Suitable salts of azelaic acid according to the invention are selected from the azelates and hydrogen azelates of alkali metal ions, alkaline earth metal ions, and the ions of basic amino acids, such as arginine, lysine, and histidine, and in particular of lithium, sodium, potassium, magnesium, and calcium ions, and mixtures of these salts.

Suitable salts of sebacic acid according to the invention are selected from the sebacates and hydrogen sebacates of alkali metal ions, alkaline earth metal ions, and the ions of basic amino acids, such as arginine, lysine, and histidine, and in particular of lithium, sodium, potassium, magnesium, and calcium ions, and mixtures of these salts.

Salts of particularly preferred maleic acid suitable according to the invention are selected from the maleates and hydrogen maleates of alkali metal ions and alkaline earth metal ions, and in particular of lithium, sodium, potassium, magnesium, and calcium ions, and mixtures of these salts.

Salts of preferred fumaric acid suitable according to the invention are selected from the fumarates and hydrogen fumarates of alkali metal ions and alkaline earth metal ions, and in particular of lithium, sodium, potassium, magnesium, and calcium ions, and mixtures of these salts.

Salts of preferred D-tartaric acid suitable according to the invention are selected from the tartrates and hydrogen tartrates of alkali metal ions and alkaline earth metal ions, and in particular of lithium, sodium, potassium, magnesium, and calcium ions, and mixtures of these salts.

Salts of preferred L-tartaric acid suitable according to the invention are selected from the tartrates and hydrogen tartrates of alkali metal ions and alkaline earth metal ions, and in particular of lithium, sodium, potassium, magnesium, and calcium ions, and mixtures of these salts.

Salts of preferred meso-tartaric acid suitable according to the invention are selected from the tartrates and hydrogen tartrates of alkali metal ions and alkaline earth metal ions, and in particular of lithium, sodium, potassium, magnesium, and calcium ions, and mixtures of these salts.

Racemic acid, which is particularly preferred in accordance with the invention, is the racemic mixture of D-tartaric acid and L-tartaric acid. Salts of racemic acid suitable according to the invention are selected from the tartrates and hydrogen tartrates of alkali metal ions and alkaline earth metal ions, and in particular of lithium, sodium, potassium, magnesium, and calcium ions, and mixtures of these salts.

Salts of alpha-ketoglutaric acid suitable according to the invention are selected from the alpha-ketoglutarates and alpha-keto hydrogen glutarates of alkali metal ions and alkaline earth metal ions, and in particular of lithium, sodium, potassium, magnesium, and calcium ions, and mixtures of these salts.

Salts of beta-ketoglutaric acid suitable according to the invention are selected from the beta-ketoglutarates and beta-keto hydrogen glutarates of alkali metal ions and alkaline earth metal ions, and in particular of lithium, sodium, potassium, magnesium, and calcium ions, and mixtures of these salts.

Salts of oxaloacetic acid suitable according to the invention are selected from the oxaloacetates and oxalo hydrogen acetates of alkali metal ions and alkaline earth metal ions, and in particular of lithium, sodium, potassium, magnesium, and calcium ions, and mixtures of these salts.

Preferred oxidative hair treatment agents according to the invention contain the at least one dicarboxylic acid having 2 to 10 carbon atoms, which is preferably selected from succinic acid, oxalic acid, malonic acid, adipic acid, pimelic acid, suberic acid, azelaic acid, sebacic acid, malic acid, D-tartaric acid, L-tartaric acid, meso-tartaric acid, racemic acid, alpha-ketoglutaric acid, beta-ketoglutaric acid, oxaloacetic acid, maleic acid, and fumaric acid and/or at least one salt of these acids, in a total amount, converted to the mass of free dicarboxylic acid, of 0.2 to 4.0 wt %, preferably 0.33 to 3.0 wt %, particularly preferably 0.5 to 2.0 wt %, and exceptionally preferably 0.7 to 1.0 wt %, in each case relative to the weight of the oxidative hair treatment agent.

Other preferred oxidative hair treatment agents according to the invention comprise succinic acid and/or at least one salt of succinic acid in a total amount, converted to the mass of free dicarboxylic acid, of 0.2 to 4.0 wt %, preferably 0.33 to 3.0 wt %, particularly preferably 0.5 to 2.0 wt %, and exceptionally preferably 0.7 to 1.0 wt %, in each case relative to the weight of the oxidative hair treatment agent.

Other preferred oxidative hair treatment agents according to the invention comprise malic acid and/or at least one salt of malic acid in a total amount, converted to the mass of free dicarboxylic acid, of 0.2 to 4.0 wt %, preferably 0.33 to 3.0 wt %, particularly preferably 0.5 to 2.0 wt %, and exceptionally preferably 0.7 to 1.0 wt %, in each case relative to the weight of the oxidative hair treatment agent.

Other preferred oxidative hair treatment agents according to the invention comprise malonic acid and/or at least one salt of malonic acid in a total amount, converted to the mass of free dicarboxylic acid, of 0.2 to 4.0 wt %, preferably 0.33 to 3.0 wt %, particularly preferably 0.5 to 2.0 wt %, and exceptionally preferably 0.7 to 1.0 wt %, in each case relative to the weight of the oxidative hair treatment agent.

Particularly preferred oxidative hair treatment agents according to the invention further contain at least one or more hydrophilic thickeners-preferably selected from polysaccharides that can be chemically and/or physically modified. Compounds from the group of polysaccharides are particularly preferred according to the invention as hydrophilic thickeners, since the basic structures of the polysaccharides are of natural origin and biodegradable. Preferred hydrophilic polysaccharide thickeners are selected from celluloses, cellulose ethers of C1-C4 alcohols, cellulose esters, xanthan gum, alginic acids (and their corresponding physiologically tolerated salts, alginates), agar agar (with the polysaccharide agarose present in agar agar as main constituent), starch fractions and starch derivatives such as amylose, amylopectin, and dextrins, karaya gum, locust bean gum, gum arabic, pectins, dextrans, and guar gum, and mixtures thereof.

Preferred cellulose ethers of C1-C4 alcohols and cellulose esters according to the invention are selected from methyl celluloses, ethyl celluloses, hydroxyalkyl celluloses (such as hydroxyethyl cellulose), methyl hydroxyalkyl celluloses, and carboxymethyl celluloses (e.g., those with the INCI name, Cellulose Gum), and the physiologically tolerated salts thereof.

In preferred embodiments, carboxymethyl cellulose (preferably carboxymethyl cellulose with the INCI name, Cellulose Gum) is contained as hydrophilic thickener in view of a reliable viscosity adjustment and residue-free application to keratin fibers and the scalp. Carboxymethyl cellulose can be contained in a preferred embodiment as the sole hydrophilic thickener. Particular preference is given to a combination of carboxymethyl cellulose and hydroxyethyl cellulose.

A combination of carboxymethyl cellulose and xanthan (preferably xanthan with the INCI name, Xanthan Gum) can also be preferred according to the invention.

Particularly preferred oxidative hair treatment agents according to the invention contain at least one hydrophilic thickener in a total amount of 0.1 to 5 wt %, preferably 0.5 to 4 wt %, more preferably 1 to 3.5 wt %, and very particularly preferably 2.5 to 3.4 wt %, in each case relative to the weight of the oxidative hair treatment agent.

In a further preferred embodiment of the present invention, the oxidative hair treatment agent according to the invention contains, in each case relative to the weight thereof, 0.1 to 4 wt %, and preferably 1 to 2.5 wt % carboxymethyl cellulose.

In a further preferred embodiment of the present invention, the oxidative hair treatment agent according to the invention contains, in each case in relation to the weight, 0.1 to 3 wt %, preferably 0.2 to 1.0 wt %, and more preferably 0.25 to 0.5 wt % hydroxyethyl cellulose.

Furthermore, the oxidative hair treatment agent according to the invention or the oxidative hair treatment agent preferred according to the invention can contain at least one direct dye. These are dyes which are drawn directly onto the hair and do not require an oxidizing process to form the color. To dull undesirable residual color impressions caused by melanin degradation products—in particular, in the red or blue spectrum—certain direct dyes of the complementary colors are particularly preferably contained. Direct dyes are usually nitrophenylene diamines, nitroaminophenols, azo dyes, anthraquinones or indophenols. Direct dyes can be anionic, cationic, or non-ionic. The respective direct dyes are preferably present in an amount of 0.001 to 2 wt %, relative to the weight of the oxidative hair treatment agent.

Preferred anionic direct dyes are the compounds known under the international names or trade names, Acid Yellow 1, Yellow 10, Acid Yellow 23, Acid Yellow 36, Acid Orange 7, Acid Red 33, Acid Red 52, Pigment Red 57:1, Acid Blue 7, Acid Green 50, Acid Violet 43, Acid Black 1, Acid Black 52, bromophenol blue, and tetrabromophenol blue. Preferred cationic direct dyes include cationic triphenylmethane dyes, e.g., Basic Blue 7, Basic Blue 26, Basic Violet 2, and Basic Violet 14, aromatic systems which are substituted with a quaternary nitrogen group, e.g., Basic Yellow 57, Basic Red 76, Basic Blue 99, Basic Brown 16, and Basic Brown 17, cationic anthraquinone dyes, such as HC Blue 16 (Bluequat B), as well as direct dyes containing a heterocyclic compound having at least one quaternary nitrogen atom, and in particular Basic Yellow 87, Basic Orange 31, and Basic Red 51. The cationic direct dyes sold under the Arianor trademark are likewise cationic direct dyes preferred in accordance with the invention. In particular, non-ionic nitro dyes and quinone dyes and neutral azo dyes are suitable as non-ionic direct dyes. Preferred non-ionic direct dyes are the compounds known under the international names or trade names, HC Yellow 2, HC Yellow 4, HC Yellow 5, HC Yellow 6, HC Yellow 12, HC Orange 1, Disperse Orange 3, HC Red 1, HC Red 3, HC Red 10, HC Red 11, HC Red 13, HC Red BN, HC Blue 2, HC Blue 11, HC Blue 12, Disperse Blue 3, HC Violet 1, Disperse Violet 1, Disperse Violet 4, Disperse Black 9, and also 1,4-diamino-2-nitrobenzene, 2-amino-4-nitrophenol, 1,4-bis(2-hydroxyethyl)amino-2-nitrobenzene, 3-nitro-4-(2-hydroxy-ethyl)aminophenol, 2-(2-hydroxyethyl)amino-4,6-dinitrophenol, 4-[(2-hydroxyethyl)amino]-3-nitro-1-methylbenzene, 1-amino-4-(2-hydroxyethyl)amino-5-chloro-2-nitrobenzene, 4-amino-3-nitrophenol, 1-(2′-ureidoethyl)amino-4-nitrobenzene, 2-[(4-amino-2-nitrophenyl)amino]benzoic acid, 6-nitro-1,2,3,4-tetrahydroquinoxaline, 2-hydroxy-1,4-naphthoquinone, picramic acid and salts thereof, 2-amino-6-chloro-4-nitrophenol, 4-ethylamino-3-nitrobenzoic acid, and 2-chloro-6-ethylamino-4-nitrophenol. A combination of tetrabromophenol blue and Acid Red 92 is contained very particularly preferably in accordance with the invention.

A further subject matter of the present invention is a method for lightening keratin fibers or dyeing keratin fibers in a lightening manner, and in particular hair, in which an oxidative hair treatment agent (B) according to the invention or an oxidative hair treatment agent that is preferred according to the invention is mixed with an oxidation composition (Ox), which, in each case in relation to its weight, contains 50-96 wt %, preferably 70-93 wt %, and particularly preferably 80-90 wt % water and 0.5-20 wt % hydrogen peroxide and also contains at least one pH adjuster in such an amount that the oxidation composition has a pH value in the range of 1.5 to 5.5 at 20° C., is applied directly thereafter to the keratin-containing fibers, is left on the fibers for 5 to 60 minutes, and then the fibers are rinsed with water and the bleaching paste is optionally washed out using a surfactant-containing cleansing agent, wherein the oxidative hair treatment agent (B) and the oxidation composition (Ox) are preferably mixed with one another in a weight-based ratio (B):(Ox) of 0.2-2, particularly preferably 0.3-1.5, and more preferably 0.5-1.

The oxidation composition (Ox) used in the lightening method according to the invention contains fundamentally water and hydrogen peroxide. The concentration of hydrogen peroxide is determined on the one hand by the legal requirements, and on the other by the desired effect. It amounts to 0.5 to 20 wt %, preferably 3 to 12 wt %, and particularly preferably 6 to 9 wt % hydrogen peroxide (calculated as 100% H₂O₂), in each case relative to the weight of the oxidation composition (Ox).

To stabilize the hydrogen peroxide, the oxidation composition (Ox) preferably has an acidic pH value, and in particular a pH value in the range of 1.5 to 5.5, measured at 20° C. To stabilize the hydrogen peroxide, complexing agents, preservatives, stabilizers, and/or buffer substances are also preferred.

According to the invention, the oxidative hair treatment agent (B) is preferably composed such that the mixture with the aforementioned oxidation composition (Ox), i.e., the ready-to-use lightening agent, and in particular the bleaching agent, has an alkaline pH value, preferably a pH value of 8.0 to 11.5, preferably in the range of 8.5 to 11.0, particularly preferably 9.0 to 10.5, and exceptionally preferably 9.5 to 10.0, each measured at 20° C.

Oxidation compositions (Ox) used particularly preferably according to the invention also contain at least one oil and/or at least one fatty component having a melting point in the range of 23-110° C., preferably in a total amount of 0.1-60 wt %, particularly preferably 0.5-40 wt %, and exceptionally preferably 2-24 wt %, in each case relative to the weight of the oxidation composition (Ox) used with particular preference according to the invention.

The oils suitable for the oxidation compositions (Ox) preferably used according to the invention are largely the same oils as those disclosed above as being suitable de-dusting agents for bleaching powders and as carrier oils for bleaching pastes.

Fatty components that are preferably used according to the invention in the oxidation compositions (Ox) and have a melting point in the range of 23 to 110° C. are selected from linear saturated 1-alkanols having 12 to 30 carbon atoms, preferably in a total amount of 0.1 to 8 wt %, and particularly preferably 3.0 to 6.0 wt %, in each case relative to the weight of the oxidation composition (Ox) used according to the invention.

The at least one linear saturated 1-alkanol having 12-30 carbon atoms is preferably selected from lauryl alcohol, myristyl alcohol, cetyl alcohol, stearyl alcohol, arachidyl alcohol, and behenyl alcohol and also from mixtures of these 1-alkanols, and particularly preferably from cetyl alcohol, stearyl alcohol, and cetyl alcohol/stearyl alcohol mixtures.

Oxidation compositions (Ox) that are used with preference according to the invention also contain, in each case relative to their weight, at least one linear, saturated 1-alkanol having 12-30 carbon atoms in a total amount of 0.1 to 8 wt %, and preferably in a total amount of 2 to 6 wt %, wherein at least one 1-alkanol is present, selected from cetyl alcohol, stearyl alcohol, and cetyl alcohol/stearyl alcohol mixtures.

Other oxidation compositions (Ox) preferably used according to the invention contain at least one fatty component having a melting point in the range of 23-110° C., which is selected from esters of a saturated monovalent C₁₆-C60 alkanol and a saturated C₈-C₃₆ monocarboxylic acid, and in particular cetyl behenate, stearyl behenate, and C₂₀-C₄₀ alkyl stearate, glycerol triesters of saturated, linear C₁₂-C₃₀ carboxylic acids that may be hydroxylated, candelilla wax, carnuba wax, beeswax, saturated, linear C₁₄-C₃₆ carboxylic acids, and mixtures of the aforementioned substances.

Other oxidation compositions (Ox) preferably used according to the invention comprise at least one surfactant or at least one emulsifier, preferably in a total amount of 0.5 to 10 wt %, and preferably 1 to 5 wt %, in each case relative to the weight of the oxidation composition (Ox) used according to the invention.

Surfactants and emulsifiers in the sense of the present application are amphiphilic (bifunctional) compounds that consist of at least one hydrophobic and at least one hydrophilic molecule part. The hydrophobic functional group is preferably a hydrocarbon chain having 8-28 carbon atoms, which can be saturated or unsaturated, linear or branched. This C₈-C₂₈ alkyl chain is particularly preferably linear. The basic properties of the surfactants and emulsifiers are oriented absorption at boundary surfaces and also the aggregation to micelles and the formation of lyotropic phases.

Anionic, non-ionic, and cationic surfactants are particularly suitable according to the invention. However, zwitterionic and amphoteric surfactants are also very suitable according to the invention.

All anionic surface-active substances that are suitable for use on the human body are suitable as anionic surfactants in the compositions according to the invention. These are characterized by a water-soluble-making anionic group, such as a carboxylate, sulfate, sulfonate, or phosphate group and a lipophilic alkyl group having 8 to 30 C atoms. In addition, glycol ether or polyglycol ether groups, ester, ether and amide groups, and hydroxyl groups can be contained in the molecule. Examples of suitable anionic surfactants are linear and branched fatty acids having 8 to 30 C atoms (soaps), alkylether carboxylic acids, acyl sarcosides, acyl taurides, acyl isethionates, sulfosuccinic acid monoesters and dialkylesters and sulfosuccinic acid mono-alkylpolyoxyethyl esters, linear alkane sulfonates, linear alpha-olefin sulfonates, alkylsulfates and alkylether sulfates, and also alkyl and/or alkenyl phosphates. Preferred anionic surfactants are alkyl sulfates, alkylether sulfates and alkylether carboxylic acids each having 10 to 18 C atoms, and preferably 12 to 14 C atoms in the alkyl group and up to 12 glycolether groups, and preferably 2 to 6 glycol ether groups in the molecule. Examples of such surfactants are the compounds with the INCI names, Sodium Laureth Sulfate, Sodium Lauryl Sulfate, Sodium Myreth Sulfate, or Sodium Laureth Carboxylate.

Surface-active compounds that carry, in the molecule, at least one quaternary ammonium group and at least one carboxylate, sulfonate, or sulfate group are referred to as zwitterionic surfactants. Particularly suitable zwitterionic surfactants are the so-called betaines, such as the N-alkyl-N,N-dimethylammonium glycinates, e.g., cocoalkyl dimethylammonium glycinate; the N-acylaminopropyl-N,N-dimethylammonium glycinates, e.g., cocoacylaminopropyl dimethylammonium glycinate; and 2-alkyl-3-carboxymethyl-3-hydroxyethyl imidazolines each having 8 to 18 C atoms in the alkyl or acyl group, and cocoacylaminoethyl hydroxyethyl carboxymethyl glycinate. A preferred zwitterionic surfactant is the fatty acid amide derivative known under the INCI name, Cocamidopropyl Betaine.

Amphoteric surfactants are understood to be surface-active compounds which, in addition to a C₈-C₂₄ alkyl or acyl group, also contain at least one free amino group and at least one —COOH— or —SO₃H group in the molecule and are capable of forming inner salts. Examples of suitable amphoteric surfactants are N-alkylglycines, N-alkylpropionic acids, N-alkylaminobutyric acids, N-alkyliminodipropionic acids, N-hydroxyethyl-N-alkylamidopropylglycines, N-alkyltaurines, N-alkylsarcosines, 2-alkyl aminopropionic acids, and alkyl amino acetic acids each having 8 to 24 C atoms in the alkyl group. Particularly preferred amphoteric surfactants are N-coco alkylamino propionate, coco acylamino ethylamino propionate, and C₁₂-C₁₈ acyl sarcosine.

Non-ionic surfactants contain, as a hydrophilic group, for example, a polyol group, a polyalkylene glycol ether group, or a combination of polyol group and polyglycol ether group. Such compounds are, for example, addition products of 4 to 50 mol ethylene oxide and/or 0 to 5 mol propylene oxide to linear and branched fatty alcohols, to fatty acids, and to alkyl phenols, in each case having 8 to 20 C atoms in the alkyl group, ethoxylated mono-, di-, and triglycerides, such as glycerol monolaurate+20 ethylene oxide, and glycerol monostearate+20 ethylene oxide, sorbitol fatty acid ester and addition products of ethylene oxide to sorbitol fatty acid ester such as polysorbate (Tween 20, Tween 21, Tween 60, Tween 61, Tween 81), addition products of ethylene oxide to fatty acid alkanolamides and fatty amines, and alkylpolyglycosides. In particular, C₈-C₂₂ alkylmono- and -oligoglycosides and ethoxylated analogues thereof and also ethylene oxide addition products to saturated or unsaturated linear fatty alcohols each having 2 to 30 mol ethylene oxide per mol of fatty alcohol are suitable as non-ionic surfactants.

Other oxidation compositions used with preference according to the invention are characterized in that the at least one anionic surfactant is selected from alkyl sulfates, alkyl ether sulfates, and alkyl ether carboxylic acids each having 10 to 18 C atoms, and preferably 12 to 14 C atoms, in the alkyl group, and up to 12, and preferably 2 to 6, glycol ether groups, in the molecule.

Other oxidation compositions used with preference according to the invention are characterized in that at least one non-ionic surfactant, selected from ethylene oxide addition products to saturated or unsaturated linear fatty alcohols each having 2 to 30 mol of ethylene oxide per mol of fatty alcohol, and at least one anionic surfactant, selected from alkyl sulfates, alkylether sulfates, and alkyl ether carboxylic acids, each having 10 to 18 C atoms, and preferably 12 to 14 C atoms, in the alkyl group and up to 12, and preferably 2 to 6, glycol ether groups, in the molecule are contained, wherein the ratio by weight of the totality of all anionic surfactants to the totality of all non-ionic surfactants particularly preferably lies in the range of 5-50, and preferably 10-30.

All cationic surface-active substances that are suitable for use on the human body are suitable in principle as cationic surfactants in oxidation compositions (Ox) used with preference according to the invention. These are characterized by at least one water-soluble-making cationic group, such as a quaternary ammonium group, or by at least one water-soluble-making, cationizable group, such as an amine group, and also at least one (lipophilically-acting) alkyl group having 6 to 30 C atoms or at least one (lipophilically-acting) imidazole group or at least one (lipophilically-acting) imidazyl alkyl group.

Oxidation compositions (Ox) used with particular preference according to the invention contain at least one cationic surfactant, which is preferably selected from quaternary ammonium compounds having at least one C8-C24 alkyl group, esterquats and amidoamines each having at least one C8-C24 acyl group, and mixtures hereof. Preferred quaternary ammonium compounds having at least one C8-C24 alkyl functional group are ammonium halides, and in particular chlorides and ammonium alkyl sulfates, such as methosulfates or ethosulfates, such as C8-C24 alkyl trimethyl ammonium chlorides, C8-C24 dialkyl dimethyl ammonium chlorides, and C8-C24 trialkyl methyl ammonium chlorides, e.g., cetyl trimethyl ammonium chloride, stearyl trimethyl ammonium chloride, distearyl dimethyl ammonium chloride, lauryl dimethyl ammonium chloride, lauryl dimethyl benzyl ammonium chloride, and tricetyl methyl ammonium chloride, and the imidazolium compounds known under the INCI names, Quaternium-27, Quaternium-83, Quaternium-87, and Quaternium-91. The alkyl chains of the above-mentioned surfactants preferably have 8 to 24 carbon atoms.

Esterquats are cationic surfactants which contain both at least one ester function and at least one quaternary ammonium group as structural element and also at least one C8-C24 alkyl group or C8-C24 acyl group. Preferred esterquats are quaternized ester salts of fatty acids with triethanolamine, quaternized ester salts of fatty acids with diethanolalkylamines, and quaternized ester salts of fatty acids with 1,2-dihydroxypropyldialkylamines. N.N-bis(2-palmitoyloxyethyl)dimethylammonium chloride, distearylethyl dimonium methosulfate and distearoylethyl hydroxyethylmonium methosulfate are preferred examples of such esterquats.

The alkylamidoamines are usually produced by the amidation of natural or synthetic C8-C24 fatty acids and fatty acid cuts with di-(C1-C3)alkylaminoamines. A compound from this substance group which is particularly suitable according to the invention is stearamidopropyl dimethylamine.

Oxide compositions (Ox) used with particular preference according to the invention contain at least one cationic surfactant in a total amount of 0.01-5 wt %, preferably 0.1-3 wt %, and particularly preferably 0.3-2 wt %, in each case in relation to the weight of the oxidation composition (Ox) used in accordance with the invention.

A further subject of the present invention is a multi-component packaging unit (kit-of-parts) for lightening keratinic fibers which contains at least two components packaged separately from one another and which is characterized in that
i) the first component (B) is an oxidative hair treatment agent according to the invention or preferred according to the invention,
ii) the second component (Ox) is an oxidizing composition that contains, in each case relative to its weight, 50-96 wt %, preferably 70-93 wt %, and particularly preferably 80-90 wt % water and 0.5-20 wt % hydrogen peroxide and also contains at least one pH setting agent in such an amount that the oxidation composition has a pH value in the range of 1.5 to 5.5, measured at 20° C.,
wherein components (B) and (Ox) are preferably present in a weight-based ratio (B):(Ox) to one another of 0.2-2, particularly preferably 0.3-1.5, more preferably 0.5-1, and exceptionally preferably 0.5-0.6.

The ready-to use mixtures of an oxidative hair treatment agent according to the invention or preferred according to the invention having one of the aforementioned oxidation compositions (Ox) preferably have a viscosity in the range of 15,000 to 100,000 mPas, and particularly preferably 20,000 to 85,000 mPas, each measured at 20° C. using a Brookfield viscosimeter Type DV-II+, spindle 5 with a speed of 4 revolutions/minute. A viscosity in this range means that the ready-for-use agent can be easily applied and also has such a flow behavior that this guarantees, for the agent, a sufficiently long time of action at the site of action on the keratin fibers.

A multi-component packaging unit comprises several individual components that are packaged separately from one another, and also a common packaging for these components—for example, a collapsible box. The components are provided therein, each separated into different containers. In the context of the present invention, a container is understood to mean a wrapping which is present in the form of an optionally re-closable bottle, a tube, a can, a bag, a sachet, or a similar wrapping. According to the invention, the wrapping material is not subject to any limitations. Preferably, however, these are enclosures made of glass or plastic material.

In addition, the packaging unit can comprise application aids, such as combs, hairbrushes or paintbrushes, personal protective clothing, and in particular disposable gloves, and a set of instructions.

The time of action is preferably 5 to 60 min, in particular 5 to 50 min, and particularly preferably 10 to 45 min. During the time of action of the agents upon the fibers, it may be advantageous to assist the lightening process by supply of heat. A phase of action at room temperature likewise accords with the invention. In particular, the temperature during the time of action is between 20° C. and 40° C., and in particular between 25° C. and 38° C. The agents already yield good treatment results at physiologically tolerated temperatures of less than 45° C.

After the end of the lightening process, all components located on the keratin fibers are rinsed from the hair using water or a surfactant-containing cleansing agent. Here, commercially available shampoo can be used in particular as cleansing agent, wherein it is then possible in particular to dispense with the cleansing agent and to carry out the rinsing process using tap water when the lightening agent has a higher surfactant content.

That said with regard to the oxidative hair treatment agents according to the invention and preferred according to the invention also applies, mutatis mutandis, to the multi-component packaging units (kits-of-parts) according to the invention and preferred according to the invention.

That said with regard to the oxidative hair treatment agents according to the invention preferred according to the invention also applies, mutatis mutandis, to the methods according to the invention and preferred according to the invention for lightening and/or changing the color of keratin fibers.

That said with regard to the oxidation compositions according to the invention and preferably used according to the invention also applies, mutatis mutandis, to the multi-component packaging units (kits-of-parts) according to the invention and preferred according to the invention.

That said with regard to the oxidation compositions according to the invention and preferably used according to the invention also applies, mutatis mutandis, to the methods according to the invention and preferred according to the invention for lightening and/or changing the color of keratin fibers.

That said with regard to the oxidative hair treatment agents according to the invention and preferred according to the invention also applies, mutatis mutandis, to the use according to the invention.

That said with regard to the oxidation compositions according to the invention preferably used according to the invention also applies, mutatis mutandis, to the use according to the invention.

In summary, the subject matter of the invention is described by the following points:

1. An oxidative hair treatment agent for lightening hair or for dyeing hair in a lightening manner, containing:
a) at least one oxidizing agent selected from inorganic salts of a peroxysulfuric acid, and mixtures of these salts,
b) further, at least one complexing agent selected from
b) i) complexing agents of the general formula (I),

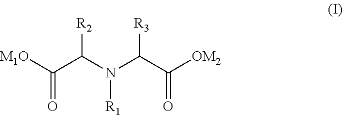

where
R1 represents a hydrogen atom, a C₁-C₆ alkyl group, a hydroxy-C₂-C₆ alkyl group, a carboxy-C₁-C₆ alkyl group, or a physiologically tolerated salt thereof,
R2, R3, independently of one another, represent a hydrogen atom, a C₁-C₆ alkyl group, a hydroxy-C₂-C₆ alkyl group, a carboxy-C₁-C₆ alkyl group, or a physiologically tolerated salt thereof, wherein R2 and R3 do not simultaneously represent a hydrogen atom and do not simultaneously represent a carboxy-C₁-C₆ alkyl group,
M1, M2, independently of one another, represent a hydrogen atom or an alkali metal ion—preferably sodium, potassium, or an ammonium ion (NH₄⁺)—and
b) ii) complexing agents of the general formula (II)

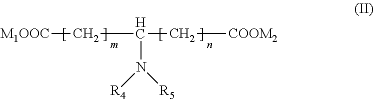

where
R4, R5, independently of one another, represent a carboxy-C₁-C₆ alkyl group or a physiologically tolerated salt thereof, a hydrogen atom, a C₁-C₆ alkyl group, or a hydroxy-C₂-C₆ alkyl group, and
m represents an integer from 0 to 6, and
n represents an integer from 0 to 6, and
M1, M2, independently of one another, represent a hydrogen atom or an alkali metal ion—preferably sodium, potassium, or an ammonium ion (NH₄⁺),
b) iii) ethylenediamine-N,N′-disuccinic acid or at least one alkali metal salt or ammonium salt of this acid,
c) further, at least one amino acid selected from alpha-amino acids, beta-amino acids, and the salts of an alpha-amino acid or beta-amino acid, and mixtures of these substances,
d) further, Polyquaternium-4, and
e) and 0 to 10 wt %, preferably 0.1 to 8 wt %, and particularly preferably 0.5 to 5 wt % water, in each case relative to the weight of the oxidative hair treatment agent, wherein no polymer is contained that comprises acrylic acid, acrylic acid amides, acrylic acid esters, methacrylic acid, methacrylic acid amides, and/or methacrylic esters as monomers.
   2. The oxidative hair treatment agent according to point 1, characterized in that the at least one oxidizing agent a) is present in a total amount of 5-85 wt %, preferably 10-70 wt %, particularly preferably 17-55 wt %, and exceptionally preferably 22-45 wt %, in each case relative to the weight of the oxidative hair treatment agent.
   3. The oxidative hair treatment agent according to point 1 or 2, characterized in that the at least one oxidizing agent a) is selected from sodium peroxydisulfate, potassium peroxydisulfate, ammonium peroxydisulfate, sodium peroxymonosulfate, potassium peroxymonosulfate, ammonium peroxymonosulfate, and mixtures of these compounds, preferably potassium peroxydisulfate-ammonium peroxydisulfate mixtures, potassium peroxydisulfate-ammonium peroxydisulfate mixtures, and potassium peroxydisulfate-sodium peroxydisulfate mixtures, particularly preferably from potassium peroxydisulfate-ammonium peroxydisulfate mixtures, and exceptionally preferably from potassium peroxydisulfate-ammonium peroxydisulfate mixtures, with an excess of potassium peroxydisulfate, which are free of sodium peroxydisulfate.
   4. The oxidative hair treatment agent according to one of points 1-3, characterized in that the at least one complexing agent b) i) of the general formula (I) is selected from methyl glycine diacetic acid, the sodium salts, potassium salts, and ammonium salts of methyl glycine diacetic acid, and mixtures of the aforementioned substances, wherein the disodium salt and the trisodium salt of methyl glycine diacetic acid are preferred, and the trisodium salt of methyl glycine diacetic acid is particularly preferred.
   5. The oxidative hair treatment agent according to one of points 1-4, characterized in that the trisodium salt of methyl glycine diacetic acid is present in a total amount of 0.1 to 5.0 wt %, preferably 0.5 to 3.0 wt %, particularly preferably 1.0 to 2.0 wt %, and exceptionally preferably 1.4 to 1.6 wt %, in each case relative to the weight of the oxidative hair treatment agent.
   6. The oxidative hair treatment agent according to one of points 1-5, characterized in that the at least one complexing agent b) ii) of the general formula (II) is selected from glutamic acid N,N-diacetic acid, the sodium salts, potassium salts, and ammonium salts of glutamic acid N,N-diacetic acid, and mixtures of the aforementioned substances, wherein the disodium salt and the trisodium salt of glutamic acid N,N-diacetic acid are preferred, and the trisodium salt of glutamic acid N,N-diacetic acid is particularly preferred.
   7. The oxidative hair treatment agent according to one of points 1-6, characterized in that the tetrasodium salt of glutamic acid N,N-diacetic acid is present as the complexing agent b) ii) in an amount of 0.1 to 5.0 wt %, preferably 0.5 to 3.0 wt %, particularly preferably 1.0 to 2.0 wt %, and exceptionally preferably 1.4 to 1.6 wt %, in each case relative to the weight of the oxidative hair treatment agent.
   8. The oxidative hair treatment agent according to one of points 1-7, characterized in that the at least one salt of ethylenediamine-N,N′-disuccinic acid is selected from sodium salts, potassium salts, and ammonium salts, and mixtures of these salts, and preferably selected from the monosodium salt, disodium salt, trisodium salt, and tetrasodium salt of ethylenediamine-N,N′-disuccinic acid, and from mixtures thereof, wherein the trisodium salt and the tetrasodium salt of ethylenediamine-N,N′-disuccinic acid are particularly preferred, and the trisodium salt of ethylenediamine-N,N′-disuccinic acid is exceptionally preferred.
   9. The oxidative hair treatment agent according to one of points 1-8, characterized in that the trisodium salt of ethylenediamine-N,N′-disuccinic acid is present as the complexing agent b) iii) in a total amount of 0.1 to 5.0 wt %, preferably 0.5 to 3.0 wt %, particularly preferably 1.0 to 2.0 wt %, and exceptionally preferably 1.4 to 1.6 wt %, in each case relative to the weight of the oxidative hair treatment agent.
   10. The oxidative hair treatment agent according to one of points 1-9, characterized in that the at least one complexing agent b) is present in a total amount of 0.1 to 5.0 wt %, preferably 0.5 to 3.0 wt %, particularly preferably 1.0 to 2.0 wt %, and exceptionally preferably 1.4 to 1.6 wt %, in each case relative to the weight of the oxidative hair treatment agent.
   11. The oxidative hair treatment agent according to one of points 1-10, characterized in that the at least one alpha-amino acid or beta-amino acid c) and/or its salt is present in a total amount, converted to the mass of free amino acid, of 0.1 to 3.0 wt %, preferably 0.2 to 1.5 wt %, particularly preferably 0.3 to 0.6 wt %, and exceptionally preferably 0.4 to 0.5 wt %, in each case relative to the weight of the oxidative hair treatment agent.
   12. The oxidative hair treatment agent according to one of points 1-11, characterized in that the at least one alpha-amino acid c) is selected from serine, arginine, the salts of these amino acids, and mixtures of the aforementioned substances, wherein mixtures of serine and arginine are particularly preferred.
   13. The oxidative hair treatment agent according to one of points 1-12, characterized in that at least one alpha-amino acid c), selected from L-arginine, L-serine, or at least one salt of these amino acids, is present in a total amount, converted to the mass of free amino acid, of 0.1 to 3.0 wt %, preferably 0.2 to 1.5 wt %, particularly preferably 0.3 to 0.6 wt %, and exceptionally preferably 0.4 to 0.5 wt %, in each case relative to the weight of the oxidative hair treatment agent.
   14. The oxidative hair treatment agent according to one of points 1-13, characterized in that Polyquaternium-4 is present in a total amount of 0.01 to 3.0 wt %, preferably 0.1 to 1.0 wt %, particularly preferably 0.1 to 0.7 wt %, and exceptionally preferably 0.3-0.5 wt %, in each case relative to the weight of the oxidative hair treatment agent.
   15. The oxidative hair treatment agent according to one of points 1-14, characterized in that at least one alkali metal or alkaline earth metal salt of a C12-C24 monocarboxylic acid, which may be saturated or unsaturated, linear or branched, is present, wherein the C12-C24 monocarboxylic acid is preferably selected from lauric acid, myristic acid, palmitic acid, palmitoleic acid, stearic acid, isostearic acid, oleic acid, elaidic acid, petroselinic acid, arachidic acid, gadoleic acid, behenic acid, erucic acid, and brassidic acid, and mixtures thereof, wherein stearic acid and palmitic acid and mixtures thereof are particularly preferred, and wherein the potassium, magnesium, and calcium salts of the C12-C24 monocarboxylic acid are particularly preferred.
   16. The oxidative hair treatment agent according to one of points 1-15, characterized in that, in each case relative to its weight, at least one alkali metal salt or alkaline earth metal salt of a C12-C24 monocarboxylic acid, which may be saturated or unsaturated, linear or branched, is present in a total amount of 0.05 to 1 wt %, preferably 0.2 to 0.6 wt %, and particularly preferably 0.3 to 0.5 wt %.
   17. The oxidative hair treatment agent according to one of points 1-16, characterized in that, in each case relative to its weight, potassium stearate is present in a total amount of 0.05 to 1 wt %, preferably 0.2 to 0.6 wt %, and particularly preferably 0.3 to 0.5 wt %.
   18. The oxidative hair treatment agent according to one of points 1-17, characterized in that 0.05 to 0.5 wt %, and preferably 0.2 to 0.4 wt % sodium hexametaphosphate is present, in each case relative to the weight of the oxidative hair treatment agent.
   19. The oxidative hair treatment agent according to one of points 1-18, characterized in that at least one dicarboxylic acid having 2 to 10 carbon atoms, selected from succinic acid, oxalic acid, malonic acid, adipic acid, pimelic acid, suberic acid, azelaic acid, sebacic acid, malic acid, D-tartaric acid, L-tartaric acid, meso-tartaric acid, racemic acid, alpha-ketoglutaric acid, beta-ketoglutaric acid, oxaloacetic acid, maleic acid, and fumaric acid, the salts of the aforementioned acids, and mixtures of the aforementioned substances, is further present, wherein the at least one dicarboxylic acid having 2 to 10 carbon atoms and/or its salt(s) in a total amount of 0.2 to 4.0 wt %, preferably 0.33 to 3.0 wt %, particularly preferably 0.5 to 2.0 wt %, and exceptionally preferably 0.7-1.0 wt % is preferably present, in each case converted to the undissociated acid and relative to the weight of the oxidative hair treatment agent.
   20. The oxidative hair treatment agent according to one of points 1-19, characterized in that at least one inorganic alkalizing agent that is solid at 20° C. and 1,013 mbar is present in a total amount of 0.1 to 50 wt %, and preferably 5 to 40 wt %, in each case relative to the weight of the oxidative hair treatment agent.
   21. The oxidative hair treatment agent according to one of points 1-20, characterized in that, in each case relative to its weight, kaolin is present in an amount of 0.05 to 5 wt %, preferably 0.5 to 4 wt %, and particularly preferably 1 to 2 wt %.
   22. The oxidative hair treatment agent according to one of points 1-21, characterized in that, relative to its weight, at least one oil selected from mineral oils, branched fatty alcohols having 8-24 carbon atoms, dialkyl ethers having 6 to 18 C atoms in the alkyl groups, and mixtures of the aforementioned substances, is present in a total amount of 0.2 to 80 wt %, preferably 1 to 70 wt %, particularly preferably 4 to 60 wt %, and exceptionally preferably 5 to 20 wt %.
   23. The oxidative hair treatment agent according to one of points 1-22, characterized in that, relative to its weight, at least one oil selected from mineral oils and 2-octyl-1-dodecanol and mixtures of the aforementioned substances is present in a total amount of 0.2 to 10 wt %, preferably 1 to 7 wt %, particularly preferably 4 to 6 wt %, and exceptionally preferably 5 to 5.5 wt %.
   24. The oxidative hair treatment agent according to one of points 1-22, characterized in that, relative to its weight, at least one oil selected from mineral oils and di-n-octyl ether and mixtures of the aforementioned substances is present in a total amount of 0.2 to 10 wt %, preferably 1 to 7 wt %, particularly preferably 4 to 6 wt %, and exceptionally preferably 5 to 5.5 wt %.
   25. A method for lightening keratin fibers or dyeing keratin fibers in a lightening manner, and in particular human hair, characterized in that an oxidative hair treatment agent (B) according to one of points 1-24 is mixed with an oxidation composition (Ox), which, in each case relative to the weight thereof, contains 50 to 96 wt %, preferably 70 to 93 wt %, and particularly preferably 80 to 90 wt % water and 0.5 to 20 wt % hydrogen peroxide, and furthermore contains at least one pH setting agent in an amount such that the oxidation composition has a pH value in the range of 1.5 to 5.5, measured at 20° C., immediately thereafter is applied to the keratin fibers and left on the fibers for 5 to 60 minutes, and, subsequently, the fibers are rinsed with water and optionally washed with a surfactant-containing cleansing agent,
wherein the oxidative hair treatment agent (B) and the oxidation composition (Ox) are preferably mixed with each other in a weight-based ratio (B):(Ox) of 0.2 to 2, particularly preferably 0.3 to 1.5, and more preferably 0.5 to 1.
   26. A method for lightening keratin fibers or dyeing keratin fibers in a lightening manner according to claim 25, characterized in that the mixture of the oxidative hair treatment agent (B) according to one of points 1-24 and the oxidation composition (Ox), and optionally after addition of a third alkalizing component, has a pH value of 8.0 to 11.5, preferably in the range of 8.5 to 11.0, particularly preferably 9.0 to 10.5, and exceptionally preferably 9.5-10.3, each measured at 20° C.
   27. A multi-component packaging unit (kit-of-parts) for lightening keratin fibers that contains at least two components packaged separately from one another and which is characterized in that
i) the first component (B) is an oxidative hair treatment agent according to one of points 1-24,
ii) the second component (Ox) is an oxidizing composition that contains, in each case relative to its weight, 50-96 wt %, preferably 70-93 wt %, and particularly preferably 80-90 wt % water and 0.5-20 wt % hydrogen peroxide, and also contains at least one pH setting agent in such an amount that the oxidation composition has a pH value in the range of 1.5 to 5.5, measured at 20° C.,
wherein the components (B) and (Ox) are preferably present in a weight-based ratio to one another (B):(Ox) of 0.2-2, particularly preferably 0.3-1.5, more preferably 0.5-1, and exceptionally preferably 0.5-0.6.

The following examples illustrate the subject matter of the invention, without limiting it thereto.

### Experimental Part

### 1. Studies on Heat Development

TABLE 1
Bleaching powder

| | 0024\01 | 0024\02 | 0024\03 | 0024\04 | 0024\05 |
|---|---|---|---|---|---|
| Basic magnesium | 10.8 | 10.8 | 10.8 | 10.8 | 10.8 |
| carbonate heavy | | | | | |
| (400 g/L) | | | | | |
| Sodium | 0.2 | 0.2 | 0.2 | 0.2 | 0.2 |
| hexametaphosphate | | | | | |
| Hydrophilic pyrogenic | 0.4 | 0.4 | 0.4 | 0.4 | 0.4 |
| silica | | | | | |
| Potassium persulfate | 32.0 | 32.0 | 32.0 | 32.0 | 32.0 |
| Ammonium persulfate | 10.0 | 10.0 | 10.0 | 10.0 | 10.0 |
| (with 0.5 wt % silica) | | | | | |
| Ultramarine blue pigment | 0.15 | 0.15 | 0.15 | 0.15 | 0.15 |
| Perfume | 0.6 | 0.6 | 0.6 | 0.6 | 0.6 |
| Paraffinum liquidum | 4.5 | 4.5 | 4.5 | 4.5 | 4.5 |
| Hydrous sodium silicate | 36.0 | 36.0 | 36.0 | 36.0 | 36.0 |
| having a molar SiO₂/ | | | | | |
| Na₂O ratio of 2.65 | | | | | |
| (Britesil C 265 from | | | | | |
| Akzo) | | | | | |
| Hydroxyethyl cellulose | 0.35 | 0.35 | 0.35 | 0.35 | 0.35 |
| Sodium carboxymethyl | 2.0 | 2.0 | 2.0 | 2.0 | 2.0 |
| cellulose | | | | | |
| Succinic acid | 1.0 | 1.0 | 1.0 | 1.0 | 1.0 |
| Lysine HCl | 0.2 | 0.2 | 0.2 | 0.2 | 0.2 |
| Arginine | 0.2 | 0.2 | 0.2 | 0.2 | 0.2 |
| Tetrasodium | 1.6 | — | — | — | — |
| iminodisuccinate | | | | | |
| (Na₄ IDS) | | | | | |
| Trisodium methyl glycine | — | 1.6 | — | — | — |
| diacetate (Na₃ MGDA) | | | | | |
| Sodium gluconate | — | | 1.6 | — | — |
| Tetrasodium glutamate | — | — | — | 1.6 | — |
| diacetate (Na₄ GLDA) | | | | | |
| Trisodium ethylene | — | — | — | — | 1.6 |
| diamine disuccinate | | | | | |
| (Na₃ EDDS) | | | | | |
| | 100.0 | 100.0 | 100.0 | 100.0 | 100.0 |

TABLE 2
Developers containing oxidizing agent

| Ingredient | Weight (wt %) |
|---|---|
| Sodium benzoate | 0.04 |
| Dipicolinic acid (2,6-dicarboxy pyridine) | 0.10 |
| Disodium pyrophosphate | 0.10 |
| Potassium hydroxide | 0.12 |
| Etidronic acid | 0.15 |
| Paraffin oil | 17.00 |
| Sodium cetearyl sulfate | 0.36 |
| Cetearyl alcohol | 3.50 |
| PEG-40 Castor Oil | 0.70 |
| Hydrogen peroxide | 9.00 |
| Water | up to 100.00 |

Each of the bleaching powders listed in Table 1 was mixed with the oil-in-water emulsion containing hydrogen peroxide according to Table 2 in a weight ratio of 1:1 and applied to hair strands doped with copper salt, which were then wrapped in aluminum foil. The temperature profile in the bleaching agent mixture was measured at regular time intervals. The results are shown in Table 3.

TABLE 3
Temperature profile [temperature in ° C.]

| Time t [minutes after | | | | | |
|---|---|---|---|---|---|
| application] | 0024\01 | 0024\02 | 0024\03 | 0024\04 | 0024\05 |
| 0 | 22 | 22 | 22 | 22 | 22 |
| 1 | 44 | 43 | 48 | 47 | 33 |
| 2 | 83 | 50 | 96 | 52 | 34 |
| 3 | 98 | 51 | 90 | 53 | 35 |
| 5 | 72 | 50 | 72 | 50 | 38 |
| 10 | 46 | 45 | 51 | 44 | 42 |
| 15 | 35 | 42 | 44 | 40 | 42 |
| 20 | 34 | 39 | 40 | 37 | 40 |
| 25 | 33 | 37 | 37 | 36 | 39 |
| 30 | 32 | 36 | 35 | 35 | 38 |
| 35 | 31 | 35 | 34 | 34 | 37 |
| 40 | 31 | 34 | 33 | 33 | 36 |
| 45 | 30.5 | 33 | 32 | 32 | 35 |

The ready-to-use bleaching agent mixtures containing tetrasodium iminodisuccinate (IDS) or sodium gluconate as a complexing agent heated up to almost 100° C. within the first three minutes. This is unsuitable for use on a human head.

By contrast, the complexing agents MGDS and GLDA used according to the invention heated up to a maximum of 51° C., which makes them to suitable substitutes for the complexing agent EDTA.

The complexing agent EDDS used according to the invention showed the lowest heating at a maximum of 42° C., which makes it an extremely suitable replacement for the complexing agent EDTA.

TABLE 4
Bleaching powder

| | 0003\51 | 0003\52 |
|---|---|---|
| Basic magnesium carbonate heavy (400 g/L) | 7.9 | 7.9 |
| Sodium hexametaphosphate | 0.4 | 0.4 |
| Hydrophilic pyrogenic silica | 0.3 | 0.3 |
| Potassium persulfate | 32.0 | 32.0 |
| Ammonium persulfate (with 0.5 wt % silica) | 10.0 | 10.0 |
| Ultramarine blue pigment | 0.15 | 0.15 |
| Perfume | 0.6 | 0.6 |
| Paraffinum liquidum | 5.2 | 4.2 |
| 2-octyl-1-dodecanol | — | 1.0 |
| Hydrous sodium silicate having a molar | 37.5 | 37.5 |
| SiO₂/Na₂O ratio of 2.65 | | |
| (Britesil C 265 from Akzo) | | |
| Hydroxyethyl cellulose | 0.25 | 0.25 |
| Sodium carboxymethyl cellulose | 2.3 | 2.3 |
| Succinic acid | 0.7 | 0.7 |
| L-serine | 0.2 | 0.2 |
| Arginine | 0.2 | 0.2 |
| Polyquaternium-4 | 0.3 | 0.3 |
| Trisodium methyl glycine diacetate | 1.4 | 1.4 |
| (Na₃ MGDA) | | |
| Potassium stearate | 0.6 | 0.6 |
| | 100.0 | 100.0 |

Each of the bleaching powders listed in Table 4 was mixed with the oil-in-water emulsion containing hydrogen peroxide according to Table 2 in a weight ratio of 1:1 in a mixing dish. The temperature profile in the bleaching agent mixture in the mixing dish was measured at regular time intervals. The results are shown in Table 5.

TABLE 5
Temperature profile [temperature in ° C.]

| Time t [minutes after application] | 0003\51 | 0003\52 |
|---|---|---|
| 0 | 22.7 | 23.5 |
| 1 | 22.9 | 24.7 |
| 2 | 24.4 | 25.7 |
| 3 | 25.0 | 26.4 |
| 5 | 25.8 | 27.1 |
| 10 | 27.5 | 28.8 |
| 15 | 29.0 | 30.4 |
| 20 | 30.5 | 31.7 |
| 25 | 31.8 | 32.9 |
| 30 | 32.9 | 34.0 |
| 35 | 33.6 | 34.9 |
| 40 | 34.1 | 35.4 |
| 45 | 34.2 | 35.6 |

### 2. Studies on Swelling Behavior

In order to optimize the bleaching powders according to the invention with regard to the swelling behavior during mixing with an emulsion (developer) containing hydrogen peroxide, the bleaching powders shown in Table 6 were prepared, and the corresponding influence of certain ingredients on the foam volume of the ready-to-use bleaching agent was investigated.

TABLE 6
Bleaching powder

| | 6-1 | 6-2 | 6-3 | 6-4 | 6-5 | 6-6 | 6-7 | 6-8 | 6-9 | 6-10 | 6-11 |
|---|---|---|---|---|---|---|---|---|---|---|---|
| Basic magnesium | 10.8 | 7.3 | 8.8 | 7.3 | 10.2 | 8.8 | 10.6 | 10.5 | 10.6 | 10.5 | 8.8 |
| carbonate heavy (400 | | | | | | | | | | | |
| g/L) | | | | | | | | | | | |
| Basic magnesium | — | — | — | — | 0.6 | — | — | — | — | — | — |
| carbonate (121 g/L) | | | | | | | | | | | |
| Sodium | 0.2 | 0.2 | 0.2 | 0.2 | 0.2 | 0.2 | 0.2 | 0.2 | 0.4 | 0.2 | 0.2 |
| hexametaphosphate | | | | | | | | | | | |
| Hydrophilic pyrogenic | 0.4 | 0.4 | 0.4 | 0.4 | 0.4 | 0.4 | 0.6 | 0.4 | 0.4 | 0.4 | 0.4 |
| silica | | | | | | | | | | | |
| Potassium persulfate | 32.0 | 32.0 | 32.0 | 32.0 | 32.0 | 32.0 | 32.0 | 32.0 | 32.0 | 32.0 | 32.0 |
| Ammonium persulfate | 10.0 | 10.0 | 10.0 | 10.0 | 10.0 | 10.0 | 10.0 | 10.0 | 10.0 | 10.0 | 10.0 |
| (with 0.5 wt % silica) | | | | | | | | | | | |
| Ultramarine blue | 0.15 | 0.15 | 0.15 | 0.15 | 0.15 | 0.15 | 0.15 | 0.15 | 0.15 | 0.15 | 0.15 |
| pigment | | | | | | | | | | | |
| Perfume | 0.6 | 0.6 | 0.6 | 0.6 | 0.6 | 0.6 | 0.6 | 0.6 | 0.6 | 0.6 | 0.6 |
| Paraffinum liquidum | 4.5 | 8.0 | 4.5 | 4.5 | 4.5 | 4.5 | 4.5 | 4.5 | 4.5 | 4.5 | 4.5 |
| Di-n-octyl ether | — | — | 2.0 | — | — | — | — | — | — | — | — |
| 2-octyl-1-dodecanol | — | — | — | 3.5 | — | — | — | — | — | — | — |
| Hydrous sodium silicates | 36.0 | 36.0 | 36.0 | 36.0 | 36.0 | 36.0 | 36.0 | 36.0 | 36.0 | 36.0 | 36.0 |
| having a molar | | | | | | | | | | | |
| SiO₂/Na₂O ratio of 2.65 | | | | | | | | | | | |
| (Britesil C 265, Akzo) | | | | | | | | | | | |
| Anhydrous sodium | — | — | — | — | — | 2.0 | — | — | — | — | — |
| metasilicate having a | | | | | | | | | | | |
| molar SiO₂/Na₂O ratio of | | | | | | | | | | | |
| 0.90-1.02 | | | | | | | | | | | |
| Hydroxyethyl cellulose | 0.35 | 0.35 | 0.35 | 0.35 | 0.35 | 0.35 | 0.35 | 0.35 | 0.35 | 0.35 | 0.35 |
| Sodium carboxymethyl | 2.0 | 2.0 | 2.0 | 2.0 | 2.0 | 2.0 | 2.0 | 2.0 | 2.0 | 2.0 | 2.0 |
| cellulose | | | | | | | | | | | |
| Succinic acid | 1.0 | 1.0 | 1.0 | 1.0 | 1.0 | 1.0 | 1.0 | 1.0 | 1.0 | 1.0 | 1.0 |
| Lysine HCl | 0.2 | 0.2 | 0.2 | 0.2 | 0.2 | 0.2 | 0.2 | 0.2 | 0.2 | 0.2 | 0.2 |
| Arginine | 0.2 | 0.2 | 0.2 | 0.2 | 0.2 | 0.2 | 0.2 | 0.2 | 0.2 | 0.2 | 0.2 |
| Polyquaternium-4 | — | — | — | — | — | — | — | 0.3 | — | — | — |
| Trisodium methyl | 1.6 | 1.6 | 1.6 | 1.6 | 1.6 | 1.6 | 1.6 | 1.6 | 1.6 | 1.6 | 1.6 |
| glycine diacetate (Na₃ | | | | | | | | | | | |
| MGDA) | | | | | | | | | | | |
| Potassium stearate | — | — | — | — | — | — | — | — | — | 0.3 | — |
| Kaolin | — | — | — | — | — | — | — | — | — | — | 2.0 |
| | 100.0 | 100.0 | 100.0 | 100.0 | 100.0 | 100.0 | 100.0 | 100.0 | 100.0 | 100.0 | 100.0 |

Each of the bleaching powders listed in Table 6 was mixed with the oil-in-water emulsion containing hydrogen peroxide according to Table 2 in a weight ratio of 1:1 and filled into a measuring cylinder. The time profile of the volume of the bleaching agent mixture was measured at regular time intervals. The pore size of the foam was assessed visually with the naked eye by trained specialist personnel. The results are shown in Table 7:

TABLE 7
Time profile of the volume of the bleaching agent mixture [mL]

| Time t [minutes | | | | | | | | | | | |
|---|---|---|---|---|---|---|---|---|---|---|---|
| after the | | | | | | | | | | | |
| mixture is | | | | | | | | | | | |
| poured into the | | | | | | | | | | | |
| measuring | | | | | | | | | | | |
| cylinder] | 6-1 | 6-2 | 6-3 | 6-4 | 6-5 | 6-6 | 6-7 | 6-8 | 6-9 | 6-10 | 6-11 |
| 0 | 50 | 50 | 50 | 50 | 50 | 52 | 52 | 50 | 50 | 50 | 50 |
| 3 | 60 | 56 | 56 | 55 | 60 | 65 | 62 | 58 | 54 | 58 | 60 |
| 5 | 68 | 64 | 64 | 63 | 67 | 73 | 68 | 65 | 60 | 64 | 67 |
| 10 | 88 | 82 | 82 | 80 | 87 | 97 | 94 | 82, | 78 | 82 | 85, |
| | | | | | | | | coarse- | | | fine- |
| | | | | | | | | pored | | | pored |
| 15 | 110 | 99 | 102 | 101 | 108, | 122, | 120 | 104 | 95 | 100, | 104 |
| | | | | | coarse- | medium- | | | | coarse- | |
| | | | | | pored | pored | | | | pored | |
| 20 | 138 | 120 | 124 | 123 | 130 | 150 | 146, | 128 | 116, | 119 | 125 |
| | | | | | | | coarse- | | fine- | | |
| | | | | | | | pored | | pored | | |
| 30 | 172 | 162 | 174 | 168, | 184 | 205 | 196 | 162 | 151 | 156 | 160 |
| | | | | fine- | | | | | | | |
| | | | | pored | | | | | | | |
| 35 | 192 | 184 | 195 | 192 | 210 | 230 | 223 | 180 | 168 | 172 | 176 |
| 40 | 210 | 202 | 218 | 214 | 234 | 248 | 242 | 196 | 183 | 188 | 192 |
| 45 | 226 | 220 | 238 | 234 | 255 | 265 | 260 | 208 | 195 | 202 | 206 |

% swelling

| | 6-1 | 6-2 | 6-3 | 6-4 | 6-5 | 6-6 | 6-7 | 6-8 | 6-9 | 6-10 | 6-11 |
|---|---|---|---|---|---|---|---|---|---|---|---|
| after 15 minutes | 220 | 198 | 204 | 202 | 216 | 235 | 231 | 208 | 190 | 200 | 208 |
| after 30 minutes | 344 | 324 | 348 | 336 | 368 | 394 | 377 | 324 | 302 | 312 | 320 |
| after 45 minutes | 452 | 440 | 476 | 468 | 510 | 510 | 500 | 416 | 390 | 404 | 412 |
| Evaluation | − | ++ | ++ | ++ | −− | −− | −− | +++ | +++ | +++ | +++ |

In the series of tests shown above, Polyquaternium-4, sodium hexametaphosphate, potassium stearate, and kaolin were identified as particularly well suited for controlling the swelling volume. Paraffin oil, di-n-octyl ether, and 2-octyl-1-dodecanol also reduce the swelling volume.

A fine-pored foam is achieved with 2-octyl-1-dodecanol, sodium hexametaphosphate, and kaolin.
On the basis of the series of tests shown in Tables 6 and 7, the bleaching powders were prepared according to Table 4.

Each of the bleaching powders listed in Table 4 was mixed with the oil-in-water emulsion containing hydrogen peroxide according to Table 2 in a weight ratio of 1:1 and filled into a measuring cylinder. The time profile of the volume of the bleaching agent mixture was measured at regular time intervals. The pore size of the foam was assessed visually with the naked eye by trained specialist personnel. The results are shown in Table 8:

TABLE 8
Time profile of the volume of the bleaching agent mixture [mL]

| Time t [minutes after the | | |
|---|---|---|
| mixture is poured into | 0003\51 | 0003\52 |
| the measuring cylinder] | [volume in mL] | [volume in mL] |
| 0 | 50 | 50 |
| 3 | 56 | 60 |
| 5 | 65 | 67 |
| 10 | 86 | 90 |
| 15 | 106 (fine-pored) | 110 (fine-pored) |
| 20 | 125 | 134 |
| 30 | 173 (medium-pored) | 181 (fine-pored) |
| 35 | 192 | 204 (medium-pored) |
| 40 | 210 | 220 |
| 45 | 225 | 236 (medium-pored) |
| | % swelling | |
| after 15 minutes | 212% | 220% |
| after 30 minutes | 346% | 362% |
| after 45 minutes | 450% | 472% |

Low swelling also means better adhesion of the mixture to a film. Large bubbles or coarse-pored foam reduce adhesion.

Partial replacement of the paraffin oil with the oil 2-octyl-1-dodecanol led to a refinement of the foam pores, resulting in improved adhesion of the bleaching agent—in particular, in the case of the film application used for strand bleaching.

The bleaching powder formulations 0003\51 and 0003\52 showed an acceptable heat development and swelling behavior.

### 3. Studies on Bleaching Power or Lightening Capacity

TABLE 9
Bleaching powder

| | | 0003\51 | 0003\52 |
|---|---|---|---|
| | | (according | (according |
| | 0018\02 | to the | to the |
| | (comparison) | invention) | invention) |
| Basic magnesium carbonate | 10.8 | 7.9 | 7.9 |
| heavy (400 g/L) | | | |
| Sodium hexametaphosphate | 0.2 | 0.4 | 0.4 |
| Hydrophilic pyrogenic silica | 0.4 | 0.3 | 0.3 |
| Potassium persulfate | 32.0 | 32.0 | 32.0 |
| Ammonium persulfate | 10.0 | 10.0 | 10.0 |
| (with 0.5 wt % silica) | | | |
| Ultramarine blue pigment | 0.15 | 0.15 | 0.15 |
| Perfume | 0.6 | 0.6 | 0.6 |
| Paraffinum liquidum | 4.5 | 5.2 | 4.2 |
| 2-octyl-1-dodecanol | — | — | 1.0 |
| Hydrous sodium silicate | 36.0 | 37.5 | 37.5 |
| having a molar | | | |
| SiO₂/Na₂O ratio of 2.65 | | | |
| (Britesil C 265 | | | |
| from Akzo) | | | |
| Hydroxyethyl cellulose | 0.35 | 0.25 | 0.25 |
| Sodium carboxymethyl | 2.0 | 2.3 | 2.3 |
| cellulose | | | |
| Succinic acid | 1.0 | 0.7 | 0.7 |
| L-serine | — | 0.2 | 0.2 |
| Arginine | 0.2 | 0.2 | 0.2 |
| Lysine HCl | 0.2 | — | — |
| Polyquaternium-4 | — | 0.3 | 0.3 |
| Trisodium methyl glycine | — | 1.4 | 1.4 |
| diacetate (Na₃ MGDA) | | | |
| Tetrasodium EDTA | 1.6 | — | — |
| Potassium stearate | | 0.6 | 0.6 |
| | 100.0 | 100.0 | 100.0 |

TABLE 10
Developers for bleaching powder which contain oxidizing agent

| Ingredient | Weight (wt % ) |
|---|---|
| Sodium benzoate | 0.04 |
| Dipicolinic acid (2,6-dicarboxy pyridine) | 0.10 |
| Disodium pyrophosphate | 0.10 |
| Potassium hydroxide | 0.12 |
| Etidronic acid | 0.15 |
| Paraffin oil | 17.00 |
| Sodium cetearyl sulfate | 0.36 |
| Cetearyl alcohol | 3.50 |
| PEG-40 Castor Oil | 0.70 |
| Hydrogen peroxide | 6.00 |
| Water | up to 100.00 |

Each of the bleaching powders listed in Table 9 was mixed with the oil-in-water emulsion containing hydrogen peroxide according to Table 10 in a weight ratio of 1:2 to form a ready-to-use bleaching agent and applied to dry hair strands of color 6-0 (light brown, Fischbach & Miller) (liquor ratio of 4 grams of ready-to-use bleaching agent per gram of hair) and rinsed out again after a time of action of 45 minutes. The ready-to-use bleaching agents each had a pH value of 10.3, measured at 20° C.

After washing and drying, the bleached strands were measured colorimetrically according to the L a b System. The colorimetric values are listed in Table 11.

TABLE 11
Colorimetric values

| | Mixture | AWM with | AWM with |
|---|---|---|---|
| | to be used | 0003\51 | 0003\52 |
| | (AWM) with | (according | (according |
| | 0018\02 | to the | to the |
| | (comparison) | invention) | invention) |
| dL | 35.76 | 36.86 | 37.72 |
| da | 3.04 | 2.27 | 2.09 |
| db | 22.31 | 22.93 | 22.89 |
| dE | 42.26 | 43.47 | 44.17 |
| Levels of Lift | 8.5 | 8.7 | 8.8 |
| (lightening levels) | | | |

The bleaching powders according to the invention achieved the same degree of lightening as the EDTA-containing bleaching powder of the prior art.

What is claimed is:

1. An oxidative hair treatment agent for lightening hair or for dyeing hair in a lightening manner, comprising:
a) at least one oxidizing agent selected from inorganic salts of a peroxysulfuric acid, and mixtures of these salts,
b) further, at least one complexing agent selected from
b)i) complexing agents of the general formula (I), 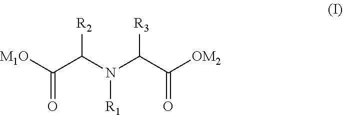
wherein R1 represents a hydrogen atom, a C₁-C₆ alkyl group, a hydroxy-C₂-C₆ alkyl group, a carboxy-C₁-C₆ alkyl group, or a physiologically tolerated salt thereof,
R2, R3, independently of one another, represent a hydrogen atom, a C₁-C₆ alkyl group, a hydroxy-C₂-C₆ alkyl group, a carboxy-C₁-C₆ alkyl group, or a physiologically tolerated salt thereof, wherein R2 and R3 do not simultaneously represent a hydrogen atom and do not simultaneously represent a carboxy-C₁-C₆ alkyl group,
M1, M2, independently of one another, represent a hydrogen atom or an alkali metal ion, and
b) ii) complexing agents of the general formula (II) 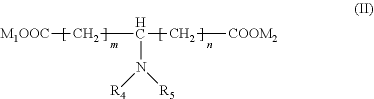
wherein R4, R5, independently of one another, represent a carboxy-C₁-C₆ alkyl group or a physiologically tolerated salt thereof, a hydrogen atom, a C₁-C₆ alkyl group, or a hydroxy-C₂-C₆ alkyl group, and
m represents an integer from 0 to 6, and
n represents an integer from 0 to 6, and
M1, M2, independently of one another, represent a hydrogen atom or an alkali metal ion,
b)iii) ethylenediamine-N,N′-disuccinic acid or at least one alkali metal salt or ammonium salt of this acid,
c) further, at least one amino acid selected from the group consisting of alpha-amino acids, beta-amino acids, and the salts of an alpha-amino acid or beta-amino acid, and mixtures thereof,
d) Polyquaternium-4, and
e) 0.1 to 10 wt %, water, in each case relative to the weight of the oxidative hair treatment agent,
wherein no polymer is contained that comprises acrylic acid, acrylic acid amides, acrylic acid esters, methacrylic acid, methacrylic acid amides, and/or methacrylic esters as monomers.

2. The oxidative hair treatment agent according to claim 1, wherein the at least one oxidizing agent a) is present in a total amount of 5-85 wt % relative to the weight of the oxidative hair treatment agent.

3. The oxidative hair treatment agent according claim 1, wherein the at least one oxidizing agent a) is selected from the group consisting of: sodium peroxydisulfate, potassium peroxydisulfate, ammonium peroxydisulfate, sodium peroxymonosulfate, potassium peroxymonosulfate, ammonium peroxymonosulfate, and mixtures of these compounds.

4. The oxidative hair treatment agent according to claim 1, wherein the at least one oxidizing agent a) is selected from the group consisting of: potassium peroxydisulfate-ammonium peroxydisulfate mixtures, potassium peroxydisulfate-ammonium peroxydisulfate mixtures, and potassium peroxydisulfate-sodium peroxydisulfate mixtures.

5. The oxidative hair treatment agent according to claim 1, wherein the at least one complexing agent b)i) of the general formula (I) is selected from methyl glycine diacetic acid, the sodium salts, potassium salts, and ammonium salts of methyl glycine diacetic acid, and mixtures of the aforementioned substances.

6. The oxidative hair treatment agent according to claim 1, wherein the at least one complexing agent b)ii) of the general formula (II) is selected from glutamic acid-N,N-diacetic acid, the sodium salts, potassium salts, and ammonium salts of glutamic acid-N,N-diacetic acid, and mixtures of the aforementioned substances.

7. The oxidative hair treatment agent according to claim 1, wherein the at least one salt of ethylenediamine-N,N′-disuccinic acid is selected from sodium salts, potassium salts, and ammonium salts, and mixtures of these salts.

8. The oxidative hair treatment agent according to claim 1, wherein the at least one complexing agent b) is present in a total amount of 0.1 to 5.0 wt % in each case relative to the weight of the oxidative hair treatment agent.

9. The oxidative hair treatment agent according to claim 1, wherein the at least one complexing agent b) is present in a total amount of 1.0 to 2.0 wt % in each case relative to the weight of the oxidative hair treatment agent.

10. The oxidative hair treatment agent according to claim 1, wherein the at least one alpha-amino acid or beta-amino acid c) and/or its salt is present in a total amount, converted to the mass of free amino acid, of 0.1 to 3.0 wt %, in each case relative to the weight of the oxidative hair treatment agent.

11. The oxidative hair treatment agent according to claim 1, wherein the at least one alpha-amino acid or beta-amino acid c) and/or its salt is present in a total amount, converted to the mass of free amino acid, of 0.3 to 0.6 wt %, in each case relative to the weight of the oxidative hair treatment agent.

12. The oxidative hair treatment agent according to claim 1, wherein the at least one alpha-amino acid c) is selected from serine, arginine, the salts of these amino acids, and mixtures of the aforementioned substances.

13. The oxidative hair treatment agent according to claim 1, wherein the Polyquaternium-4 is present in a total amount of 0.01 to 3.0 wt %, in each case relative to the weight of the oxidative hair treatment agent.

14. The oxidative hair treatment agent according to claim 1, further comprising at least one alkali metal or alkaline earth metal salt of a C12-C24 monocarboxylic acid, which may be saturated or unsaturated, linear or branched.

15. The oxidative hair treatment agent according to claim 14, wherein the at least one alkali metal or alkaline earth metal salt of C12-C24 monocarboxylic acid selected is from the group consisting of: lauric acid, myristic acid, palmitic acid, palmitoleic acid, stearic acid, isostearic acid, oleic acid, elaidic acid, petroselinic acid, arachidic acid, gadoleic acid, behenic acid, erucic acid, and brassidic acid, and mixtures thereof.

16. The oxidative hair treatment agent according to claim 1, further comprising 0.05-0.5 wt % of sodium hexametaphosphate, in each case relative to the weight of the oxidative hair treatment agent.

17. The oxidative hair treatment agent according to claim 1, further comprising at least one dicarboxylic acid having 2 to 10 carbon atoms, said at least one dicarboxylic acid having 2 to 10 carbon atoms being selected from the group consisting of succinic acid, oxalic acid, malonic acid, adipic acid, pimelic acid, suberic acid, azelaic acid, sebacic acid, malic acid, D-tartaric acid, L-tartaric acid, meso-tartaric acid, racemic acid, alpha-ketoglutaric acid, beta-ketoglutaric acid, oxaloacetic acid, maleic acid, and fumaric acid, the salts of the aforementioned acids, and mixtures of the aforementioned substances, said at least dicarboxylic acid having 2 to 10 carbon atoms and/or its salt(s) in a total amount of 0.2 to 4.0 wt %, in each case converted to the undissociated acid and relative to the weight of the oxidative hair treatment agent.

18. The oxidative hair treatment agent according to claim 1, further comprising at least one inorganic alkalizing agent that is solid at 20° C. and 1,013 mbar and is present in a total amount of 0.1 to 50 wt %, in each case relative to the weight of the oxidative hair treatment agent.

19. A method for lightening keratin fibers or dyeing keratin fibers in a lightening manner, wherein an oxidative hair treatment agent (B) according claim 1 is mixed with an oxidation composition (Ox), which, in each case relative to the weight thereof, contains 50 to 96 wt %, water and 0.5 to 20 wt % hydrogen peroxide, and furthermore contains at least one pH setting agent in an amount such that the oxidation composition has a pH value in the range of 1.5 to 5.5, measured at 20° C., immediately thereafter is applied to the keratin fibers and left on the fibers for 5 to 60 minutes, and, subsequently, the fibers are rinsed with water and optionally washed with a surfactant-containing cleansing agent,
wherein the oxidative hair treatment agent (B) and the oxidation composition (Ox) are mixed with each other in a weight-based ratio (B):(Ox) of 0.2 to 2.
